(19) United States
(12) Patent Application Publication
(10) Pub. No.: US 2017/0217872 A1
(43) Pub. Date: Aug. 3, 2017

(54) Method for the preparation of Intermediates for carboxy-fluoresceins and novel carboxy-fluorescein

(71) Applicant: Danmarks Tekniske Universitet, Kgs. Lyngby (DK)

(72) Inventors: Peter Lund Hammershøj, Brønshøj (DK); Mads Hartvig Clausen, København N (DK)

(73) Assignee: Danmarks Tekniske Universitet, Kgs. Lyngby (DK)

(21) Appl. No.: 15/330,787

(22) PCT Filed: May 6, 2015
(86) PCT No.: PCT/EP2015/059950

§ 371 Date: Nov. 7, 2016

(30) Foreign Application Priority Data
May 7, 2014 (EP) 14167285.7
Mar. 26, 2015 (EP) 15161033.4

(51) Int. Cl. C07C 65/40; C07D 311/92; C07C 51/43; C07D 311/80; C07C 51/367; C07C 51/48

(52) U.S. Cl. CPC C07C 65/40; C07C 51/367; C07C 51/48; C07C 51/43; C07D 311/80; C07D 311/92

(57) ABSTRACT

The invention provides a method for the preparation of regioisomerically pure intermediates which are useful for the preparation of carboxy-fluorescein-type compounds. Such compounds have broad applications within bio-conjugation and/or fluorescent imaging.

### FIELD OF THE INVENTION

The present invention relates to a novel method for the preparation of regioisomerically pure intermediates which are useful for the preparation of carboxy-fluorescein-type compounds. Such compounds have broad applications within bio-conjugation and/or fluorescent imaging.

### BACKGROUND OF THE INVENTION

5(6)-Carboxy-fluorescein is a well-known chromophore and mixtures of the two regioisomers can with great effort be separated into the pure regioisomers 5- and 6-carboxyfluorescein by HPLC. Burgess and co-workers [Y. Ueno, G.-S. Jiao, K. Burgess, Synthesis (Stuttg). 2004, 2591-2593] have reported a procedure using fractional crystallization in multi-gram amounts with 98% regioisomeric purity of both isomers. Separation of other regioisomeric derivatives of fluorescein has also been achieved [F. M. Rossi, J. P. Kao, Bioconjugate Chem. 1997, 8, 495-497; G.-S. Jiao, J. W. Han, K. Burgess, J. Org. Chem. 2003, 68, 8264-8267; M. Adamczyk, C. M. Chan, J. R. Fino, P. G. Mattingly, J. Org. Chem. 2000, 68, 596-601; C. C. Woodroofe, M. H. Lim, W. Bu, S. J. Lippard, Tetrahedron 2005, 61, 3097-3105.]. However, to our knowledge, a large scale synthesis without chromatographic purification to produce 100% regioisomerically pure carboxyfluoresceins has never been disclosed.

US 2002/146726 A1 discloses electrophoretic tag reagents comprising fluorescent compounds.

CN 103 012 354 A seems to disclose a method for the preparation og 5- and 6-carboxyfluorescein.

U.S. Pat. No. 4,945,171 A discloses xanthene dyes having a fused (c) benzo ring.

Sikhibhushan Dutt: CL.—A theory of Colour on the Basis of Molecular Strain. The Effect of Chromophoric Superposition, J. Chem. Soc. Vol 129, January 1926 (1926-01), p. 1171-1184, XP55136205, ISSN: 0368-1769, DOI: 10.1039/jr9262901171 discloses a number of chromophores.

U.S. Pat. No. 6,229,024 B1 discloses a method for detecting neuronal degeneration and anionic fluorescein homologue stains therefor.

U.S. Pat. No. 8,029,765 B2 discloses SMMR (Small Molecule Metabolite Reporters) for use as in vivo glucose biosensors.

U.S. Pat. No. 5,800,996 A discloses energy transfer dyes with enhanced fluorescence.

It is an object of embodiments of the invention to provide a method for the easy and cost efficient synthesis of regioisomerically pure key intermediates which are useful for the preparation of a variety of carboxy-fluoresceins including carboxy-SNAFL derivatives. By providing a method for the preparation of key intermediates which are regioisomerically pure a simple and efficient production suitable for large scale synthesis of a variety of carboxy-fluoresceins have become possible.

### SUMMARY OF THE INVENTION

It has been found by the present inventor(s) that the benzophenones 4-(2,4-dihydroxybenzoyl)isophthalic acid (6) and 2-(2,4-dihydroxybenzoyl)terephthalic acid (5) can be prepared in high regioisomerical purity by condensation of trimellitic anhydride with resorcinol with subsequent partial reversal of the condensation by hydrolysis under basic conditions, followed by acidification, isolation and fractional crystallisation of each of the target compounds.

So, in a first aspect the present invention relates to the methods defined in claim 1 and in claim 2.

In a second aspect, the invention relates to the novel carboxy-fluorescein derivatives defined in claims 12-15.

In a third aspect, the invention relates to the novel intermediates 5 and 6 defined in claim 16.

### BRIEF DESCRIPTION OF THE SCHEMES

Scheme 1. Synthetic route to regioisomerically pure 5- and 6-carboxyfluorescein (7 and 8) and mixed fluorescein derivatives 9-11.

Scheme 2. Synthetic route to mixed difluorescein derivatives 14-18.

Scheme 3. Synthesis of type [a], [b] and [c]benzoxanthenes.

### DETAILED DISCLOSURE OF THE INVENTION

### Method for the Preparation and Isolation of Compound 5 and Compound 6

One aspect of the invention relates to a method for the preparation and isolating of compound 6 and, optionally, of compound 5. The method is illustrated generally in Scheme 1.

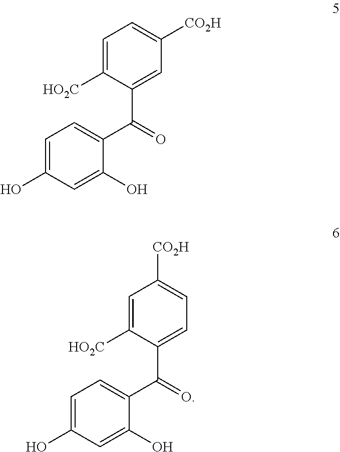 [SMILES: O=C(O)c1ccc(C(=O)O)c(C(=O)c2ccc(O)cc2O)c1 ; O=C(O)c1ccc(C(=O)c2ccc(O)cc2O)c(C(=O)O)c1]

It should be understood that the method is useful for the preparation and isolation of both compounds, but insofar that only compound 6 is of interest, compound 5 need not be isolated.

### Step (i)

In the first step of the method, a condensation product mixture is provided, being the result of a condensation reaction between trimellitic anhydride and resorcinol mediated by acid. The condensation product mixture comprises a mixture of crude 5- and 6-carboxy-fluorescein.

The method can begin from the condensation product mixture itself, or include a pre-step, in which trimellitic anhydride is reacted with resorcinol in a strong acid so as to obtain the condensation product mixture. Although not strictly necessary, the condensation product mixture is typically worked up by pouring the reaction mixture into cold water (e.g. ice water), isolation of the solid mater by filtration, refluxing in EtOH, and re-precipitation by addition of water, whereby a mixture of crude 5- and 6-carboxy-fluorescein is obtained.

Examples of acids suitable for the acid-mediated condensation reaction are methanesulfonic acid (MSA), mixtures of methanesulfonic acid and trifluoroacetic acid (TFA), e.g. an approx. 1:1 mixture of MSA and TFA, and ZnCl₂. Methanesulfonic acid is a currently preferred choice. Alternative strong acids include H₂SO₄, SnCl₄, acetic acid, H₃PO₄, HF, BF₃ and BBr₃.

The condensation reaction is conducted as previously described in the literature. Hence, typical conditions are reaction for 10-40 hours at 50-100° C., either with or without an inert atmosphere.

### Step (ii)

Subsequent to the condensation, the condensation product mixture (i.e. the crude 5- and 6-carboxy-fluorescein) is hydrolysed with a strong aqueous base at pH at least 11, typically at pH 12-14, so as to partly reverse the condensation reaction.

Examples of strong aqueous bases are 5:1/1:5 weight ratio of NaOH, KOH, LiOH, CsOH, Ca(OH)₂, Ba(OH)₂, Sr(OH)₂, NH₃ and H₂O of which 1:1 weight ratio of NaOH and H₂O is currently preferred. The skilled person will be able to select other strong aqueous bases which will achieve the desired result.

The hydrolysis is typically carried out from 1-200 hours, preferably 5-100 hours, more preferably 12-48 hours. Typical temperatures for the hydrolysis are 0-150° C., preferably 40-100° C.

In a most preferred combination of embodiments, hydrolysis is carried out using a 1:1 mixture of NaOH/H₂O at 80° C. overnight.

Step (iii)

In a subsequent step, the reaction mixture of step (ii) is acidified so as to isolate a mixture of compound 5 and compound 6.

Acidification is typically conducted by first pouring the hydrolysis reaction mixture into ice or cold water (ice water) after which a strong acid is slowly added until pH<7. Examples of strong acids are HCl, H₃PO₄, H₂CO₃, H₂SO₄, acetic acid and HNO₃, of which 12 M HCl is currently preferred. The acidification is typically conducted at 0-10° C. Acidification is usually carried out over a period of 1-4 hours.

### Step (iv)

Subsequent to the acidification, the mixture of compound 5 and compound 6 is dissolved in methanol and water is then added so as to selectively precipitate compound 6.

Crystallization is typically conducted at 0-30° C., preferably 20° C. Typical crystallisation times are 1-200 hours, preferably 24 hours. The solvent for recrystallization is typically 1-10% v/v MeOH in H2O, preferably 5% v/v.

### Step (v)

In order to isolate compound 5 and any remaining compound 6, the mother liquor from the crystallisation in step (iv) is extracted with an organic solvent, such as diethylether, ethyl acetate or dichloromethane. Of these, diethylether is preferred. The organic solvent is subsequently removed so as to obtain a dried extract. The extraction is conducted at room temperature, i.e. up to 25° C.

### Step (vi)

Steps (iv) and (v) may optionally be repeated in one or more additional cycles (e.g. 1-5 additional cycles) using the dried extract obtained in step (v) so as to crystallize out more of compound 6. Typically, 2-3 additional cycles are preferred.

Step (vii)

Insofar as isolation of compound 5 is desirable, the dried extract obtained in step (v) is dissolved in refluxing H₂O and compound 5 is precipitated. Precipitation of compound 5 suitably takes place at 0-10° C., in a time period of 1-200 hours, preferably 100 hours.

### Method for the Preparation and Isolation of Compound 13

Another aspect of the invention relates to a method for the preparation and isolation of compound 13. The method is illustrated generally in Scheme 2.

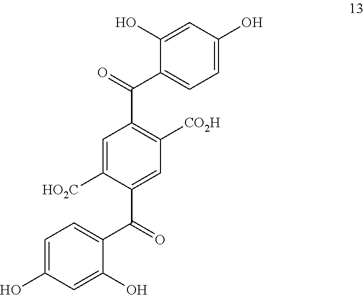 [SMILES: O=C(O)c1cc(C(=O)c2ccc(O)cc2O)c(C(=O)O)cc1C(=O)c1ccc(O)cc1O]

### Step (i)

In the first step of the method, a condensation product is provided, being the result of a condensation reaction between pyromellitic dianhydride and resorcinol in a strong acid.

The method begins with pyromellitic dianhydride that is reacted with resorcinol mediated by acid so as to obtain the condensation product. Although not strictly necessary, the condensation product is typically worked up by pouring the reaction mixture into cold water (e.g. ice water), isolation of the solid mater by filtration, refluxing in EtOH, and re-precipitation by addition of water, whereby the condensation product is obtained.

Examples of acids suitable for use in the condensation reaction are methanesulfonic acid (MSA), mixtures of methanesulfonic acid and trifluoroacetic acid (TFA), e.g. an approx. 1:1 mixture of MSA and TFA, and ZnCl₂. Methanesulfonic acid is a currently preferred choice. Alternative strong acids include H₂SO₄, SnCl₄, acetic acid, H₃PO₄, HF, BF₃ and BBr₃.

The condensation reaction is conducted as previously described in the literature. Hence, typical conditions are reaction for 10-40 hours at 50-100° C., either with or without an inert atmosphere.

### Step (ii)

Subsequent to the condensation, the condensation product is hydrolysed with a strong aqueous base at pH at least 11, typically at pH 12-14, so as to partly reverse the condensation reaction.

Examples of strong aqueous bases are 5:1/1:5 weight ratio of NaOH, KOH, LiOH, RbOH, Ca(OH)₂, Ba(OH)₂, Sr(OH)₂, NH₃ and H₂O of which 1:1 weight ratio of NaOH and H₂O is currently preferred. The skilled person will be able to select other strong aqueous bases which will achieve the desired result.

The hydrolysis is typically carried out from 1-200 hours, preferably 12-48 hours. Typical temperatures for the hydrolysis are 0-150° C., preferably 40-100° C.

In a most preferred combination of embodiments, hydrolysis is carried out using a 1:1 (v/w) mixture of NaOH/H₂O at 80° C. overnight.

Step (iii)

In a subsequent step, the reaction mixture of step (ii) is acidified so as to isolate compound 13.

Acidification is typically conducted by first pouring the hydrolysis reaction mixture into ice or cold water (ice water) after which a strong acid is slowly added. Examples of strong acids are HCl, H₃PO₄, H₂CO₃, H₂SO₄, acetic acid and HNO₃, of which 12 M HCl is currently preferred. The acidification is typically conducted at 0-10° C. Acidification is usually carried out over a period of 1-4 hours.

### Method for the Preparation of Carboxy-Fluoresceins

The compounds 5, 6 and 13 prepared according to the methods described above are useful for the preparation of a broad range of carboxy-fluoresceins (see Schemes 1 and 2).

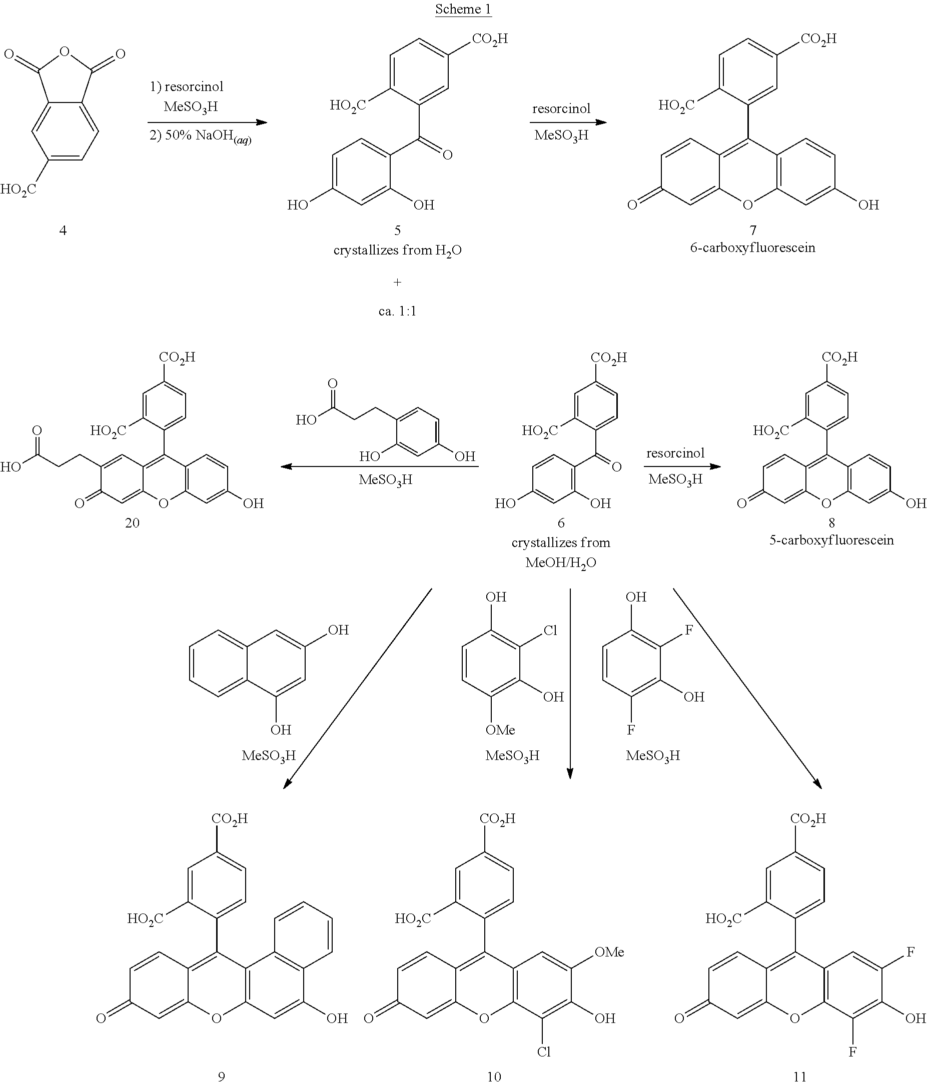

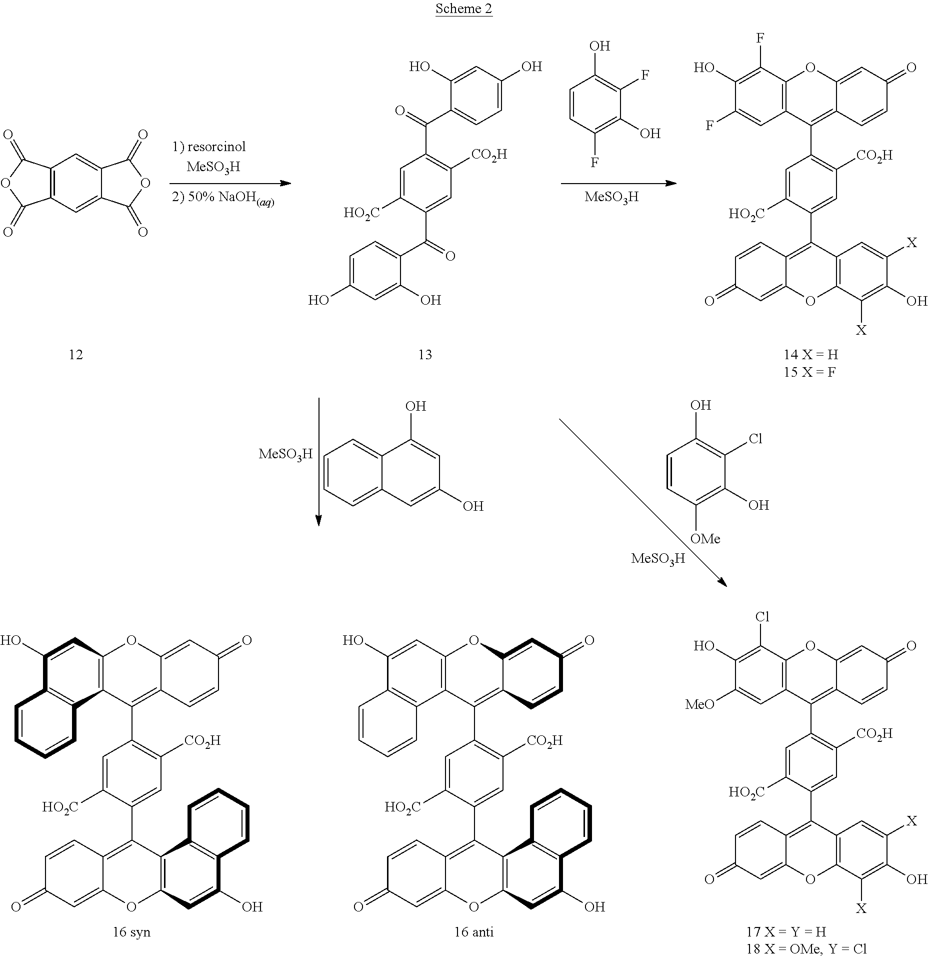

Hence, the invention also provides a method wherein compound 5 or compound 6 (e.g. obtained as described further above) is subsequently reacted with a compound of formula A

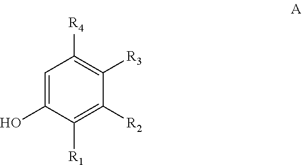

in which R₁, R₂, R₃ and R₄ are independently selected from hydrogen; halogen; hydroxyl; nitro; cyano; mercapto; —O—C₁₋₆-alkyl; —S—C₁₋₆-alkyl; cyclopropyl; —C₁₋₆-alkyl; —C₁₋₆-alkyl-CONH—R₅, —C₂₋₆-alkenyl; or —C₂₋₆-alkynyl; which —O—C₁₋₆-alkyl, —S—C₁₋₆-alkyl, cyclopropyl, —C₁₋₆-alkyl, —C₂₋₆-alkenyl or —C₂₋₆-alkynyl is optionally substituted with at least one substituent selected from halogen, hydroxyl, —COOH, nitro, cyano and mercapto; wherein R₅ is selected from the group consisting of —C₁₋₆-alkyl and —[CH₂CH₂O]ₙ, wherein n=1-10,000, wherein said —C₁₋₆-alkyl and —[CH₂CH₂O]ₙ are optionally substituted with a substituent selected from the group consisting of —NH-biotin, —C₁₋₆-alkyl-heterocycloalkyl, -DOTA, —NHCO—C₁₋₆-alkyl-heterocycloalkyl, -maleimide, —N₃, —C≡CH, —C-₁₋₆-alkyl-N₃, and —C-₁₋₆-alkyl-N(—C-₁₋₆-alkyl-heteroaryl)₂; with the additional option that any of the substituent pairs, R₁/R₂, R₂/R₃ and R₃/R₄ together with the intervening atoms may form an optionally substituted aromatic ring or ring system; in the presence of a strong acid (e.g. 99.5% pure methanesulfonic acid) so as to provide a compound of formula B

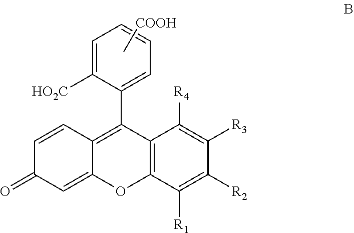

wherein R₁, R₂, R₃ and R₄ are as defined above.

Examples of strong acids suitable are methanesulfonic acid (MSA), mixtures of methanesulfonic acid and trifluoroacetic acid (TFA), e.g. an approx. 1:1 mixture of MSA and TFA, ZnCl₂. Methanesulfonic acid is a currently preferred choice. Alternative strong acids include H₂SO₄, SnCl₄, acetic acid, H₃PO₄, HF, BF₃ and BBr₃.

Also, the invention also provides a method wherein compound 13 is subsequently reacted with a compound of formula A

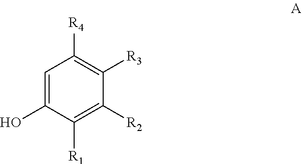

wherein R₁, R₂, R₃ and R₄ are independently selected from hydrogen; halogen; hydroxyl; nitro; cyano; mercapto; —O—C₁₋₆-alkyl; —S—C₁₋₆-alkyl; cyclopropyl; —C₁₋₆-alkyl; —C₁₋₆-alkyl-CONH—R₅, —C₂₋₆-alkenyl; or —C₂₋₆-alkynyl; which —O—C₁₋₆-alkyl, —S—C₁₋₆-alkyl, cyclopropyl, —C₁₋₆-alkyl, —C₂₋₆-alkenyl or —C₂₋₆-alkynyl is optionally substituted with at least one substituent selected from halogen, hydroxyl, —COOH, nitro, cyano and mercapto; wherein R₅ is selected from the group consisting of —C₁₋₆-alkyl and —[CH₂CH₂O]ₙ, wherein n=1-10,000, wherein said —C₁₋₆-alkyl and —[CH₂CH₂O]ₙ are optionally substituted with a substituent selected from the group consisting of —NH-biotin, —C₁₋₆-alkyl-heterocycloalkyl, -DOTA, —NHCO—C₁₋₆-alkyl-heterocycloalkyl, -maleimide, —N₃, —C≡CH, —C-₁₋₆-alkyl-N₃, and —C-₁₋₆-alkyl-N(—C-₁₋₆-alkyl-heteroaryl)₂; with the additional option that any of the substituent pairs, R₁/R₂, R₂/R₃ and R₃/R₄ together with the intervening atoms may form an optionally substituted aromatic ring or ring system;

in the presence of a strong acid (e.g. 99.5% pure methanesulfonic acid) so as to provide a compound of formula C

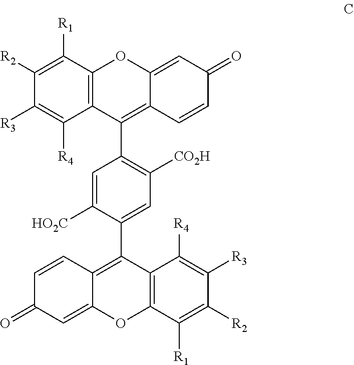

wherein R₁, R₂, R₃ and R₄ are as defined above.

Again, examples of strong acids suitable for use in this reaction are methanesulfonic acid (MSA), mixtures of methanesulfonic acid and trifluoroacetic acid (TFA), e.g. an approx. 1:1 mixture of MSA and TFA, ZnCl₂. Methanesulfonic acid is a currently preferred choice. Alternative strong acids include H₂SO₄, SnCl₄, acetic acid, H₃PO₄, HF, BF₃ and BBr₃.

Typically, in the definitions of R1, R₂, R₃ and R₄, —O—C₁₋₆-alkyl is —O—C₁₋₃-alkyl, wherein —O—C₁₋₃-alkyl is preferably —OCH₃ or —OC₂H₅. Additionally, —S—C₁₋₆-alkyl may typically be —S—C₁₋₃-alkyl, wherein —S—C₁₋₃-alkyl may preferably be —SCH₃ or —SC₂H₅. —C₁₋₆-alkyl may be methyl, ethyl, p-propyl, isopropyl, p-butyl, isobutyl, sec-butyl, tert-butyl, pentyl, isopentyl, hexyl or isohexyl. —C₁₋₆-alkyl may typically be —C₁₋₃-alkyl, wherein —C₁₋₃-alkyl may be methyl, ethyl or propyl (such as n-propyl or i-propyl). Preferably, R₂ and/or R₄ is hydroxyl, so that a 1,3-aromatic diol is included in compounds of formula A. Preferred compounds of formula A are those in which R₁ is halogen, preferably F or Cl. R₃ is preferably-O—C₁₋₃-alkyl, such as —OCH₃ or —OC₂H₅.

The term “—C₂₋₆-alkenyl” is intended to indicate a mono-, di-, or triunsaturated hydrocarbon radical comprising 2-6 carbon atoms, in particular 2-4 carbon atoms, such as 2-3 carbon atoms, e.g. vinyl, allyl, propenyl, butenyl, pentenyl or hexenyl.

The term “—C₂₋₆-alkynyl” is intended to indicate a hydrocarbon radical comprising 1-4 C—C triple bonds, e.g. 1, 2 or 3 triple bonds and 2-6 carbon atoms, the alkane chain typically comprising 2-5 carbon atoms, in particular 2-4 carbon atoms, such as 2-3 carbon atoms, e.g. ethynyl, propynyl, butynyl or pentynyl.

The term “heterocycloalkyl” is intended to include a cycloalkyl radical, wherein “cycloalkyl” indicates a saturated cycloalkane radical, comprising 3-8 carbon atoms, such as 4-7 or 3-6 carbon atoms, such as 4-6 or preferably 5-6 carbon atoms, e.g. cyclopropyl, cyclobutyl, cyclopentyl, cyclohexyl or cycloheptyl, said “heterocycloalkyl” comprising 1-7 carbon atoms, such as 1-6 carbon atoms, in particular a 4-, 5- or 6-membered ring, comprising 2-5 carbon atoms and 1-5 hetero atoms (selected from O, S and N), such as 3-5 carbon atoms and 1-3 hetero atoms, preferably 4-5 carbon atoms and 1-2 hetero atoms selected from O, S, or N, e.g. morpholino, morpholinyl, pyrrolidinyl, oxo-pyrrolidinyl, piperidino, azetidinyl, tetrahydro-furyl, tetrahydro-pyranyl, oxo-tetrahydro-furyl, oxo-oxazolidinyl, oxetanyl, dioxo-imidazolidinyl, piperidyl or piperazinyl. Preferred heterocycloalkyl radicals include pyrrolidinyl, piperazinyl and imidazolidinyl.

The term “heteroaryl” is intended to include radicals of (a) heterocyclic aromatic ring(s), comprising 1-4 heteroatoms (selected from O, S and N) and 1-10 carbon atoms, such as 1-3 heteroatoms and 1-6 carbon atoms, such as 1-3 heteroatoms and 2-5 carbon atoms, such as 1-2 heteroatoms and 3-5 carbon atoms, preferably 5- or 6-membered rings with 1-3 heteroatoms and 2-5 carbon atoms or 1-3 heteroatoms and 2-4 carbon atoms selected from O, S and N, e.g. pyridyl, thiazolyl, imidazolyl, isoxadiazolyl, [1,2,4]oxadiazolyl, oxazolyl, pyrazolyl, indolyl, thienyl, furyl, 1-benzo[b]thiophenyl, 2,3-dihydro-benzo[1,4]dioxinyl, or 2,3-dihydro-benzofuryl. Preferred heteroaryl radicals include pyridyl, 1,2,3-triazolyl and furyl.

The term “DOTA” stands for 1,4,7,10-tetraazacyclododecane-1,4,7,10-tetraacetic acid.

The term “biotin” stands for 5-[(3aS,4S,6aR)-2-oxohexahydro-1H-thieno[3,4-d]imidazol-4-yl]pentanoic acid.

The term “maleimide” stands for 2,5-pyrroledione.

The term “together with the intervening atoms may form an optionally substituted aromatic ring or ring system” is intended to mean that an aromatic ring of an aromatic ring system is fused to the benzene ring to which the substituent pairs are attached. Examples of aromatic rings are a benzene ring and a pyridine ring.

Preferably, R₃/R₄ together with the intervening atoms may form an optionally substituted aromatic ring or ring system while R₂ is hydroxy. The compound of formula A may therefore be a dihydroxynaphthalene, as illustrated in Scheme 3.

Such aromatic rings or ring systems may (or may not) be substituted with one or more substituents selected from hydrogen; halogen; hydroxyl; nitro; cyano; mercapto; —O—C₁₋₃-alkyl; —S—C₁₋₃-alkyl; cyclopropyl; —C₁₋₃-alkyl; —C₂₋₃-alkenyl; or —C₂₋₃-alkynyl.

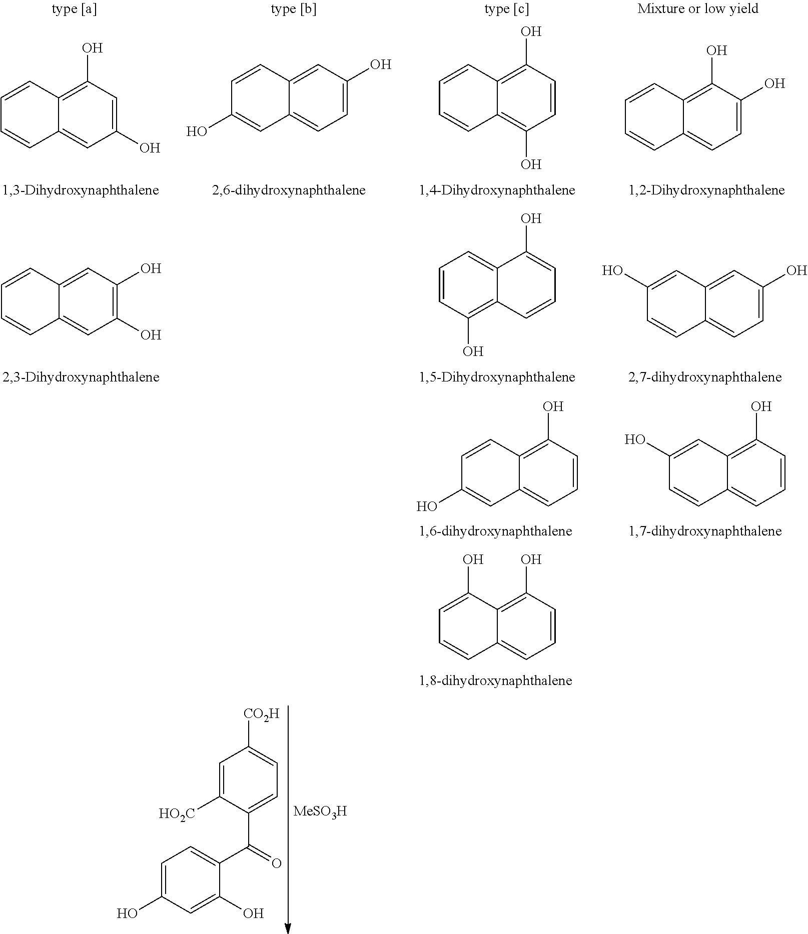

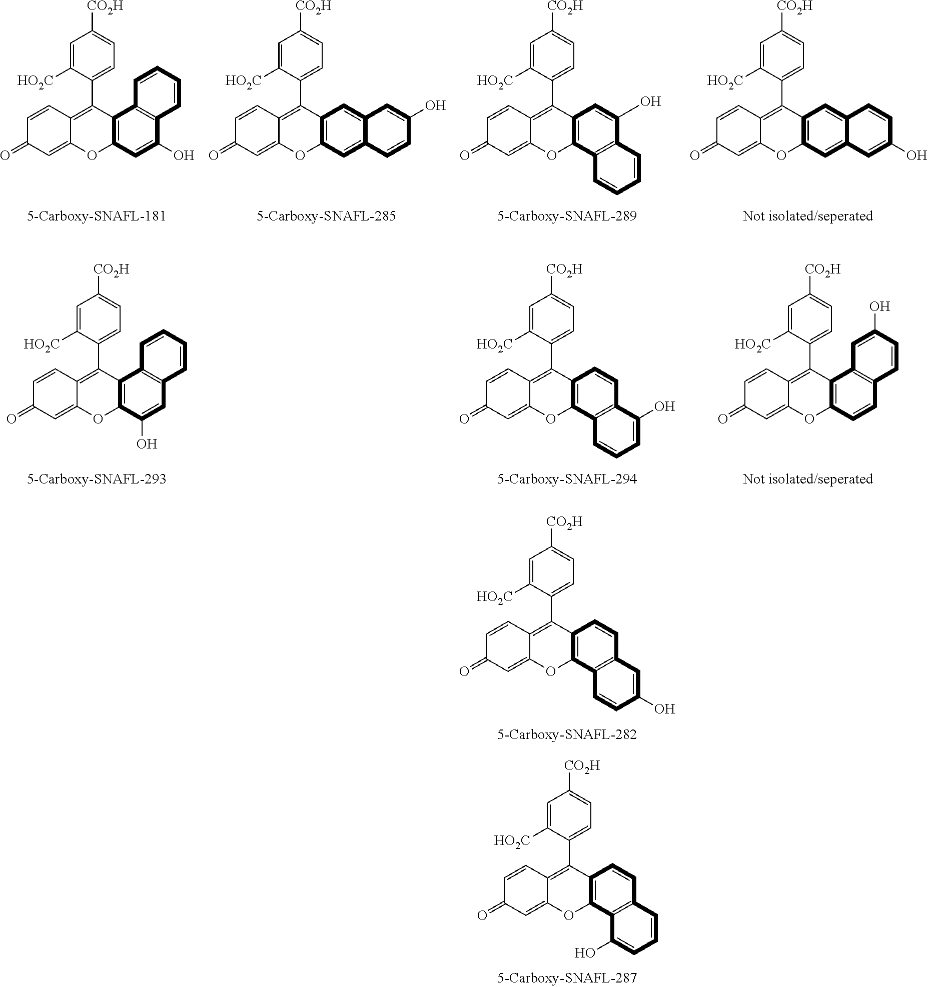

The condensation reaction between the compound of formula A and compound 5 or compound 6 or compound 13, respectively, is typically conducted for 10-40 hours at 50-100° C., either with or without an inert atmosphere.

The condensation product mixture is typically worked up by quenching the reaction (e.g. by addition of water) and the sedimented product is isolated (e.g. by centrifuging, decantation or both). Further purification steps may include recrystallization, drying, washing and chromatographic separation, as required.

It should be understood that in the preparation of the carboxy-fluoresceins of the formula B, it is not a prerequisite that the compound 5 or compound 6 or compound 13 (as the case may be) are prepared according to the method described hereinabove. The method is equally applicable when using compound 5 or compound 6 or compound 13 obtained from other sources.

The choice of the compound of formula A will be decisive for the structure of the target compound of formula B and the target compound of formula C. For instance, as illustrated in Scheme 3, A may be a dihydroxynaphthalene, such as 1,3-dihydroxynaphthalene, 2,3-dihydroxynaphthalene, 2,6-dihydroxynaphthalene, 1,4-dihydroxynaphthalene, 1,5-dihydroxynaphthalene, 1,6-dihydroxynaphthalene, 1,8-dihydroxynaphthalene, 1,2-dihydroxynaphthalene, 2,7-dihydroxynaphthalene or 1,7-dihydroxynaphthalene.

An interesting compound B derived from compound 5 is 4-(6-hydroxy-3-oxo-3H-xanthen-9-yl)isophthalic acid (8).

Some of the most interesting compounds B derived from compound 6 are:

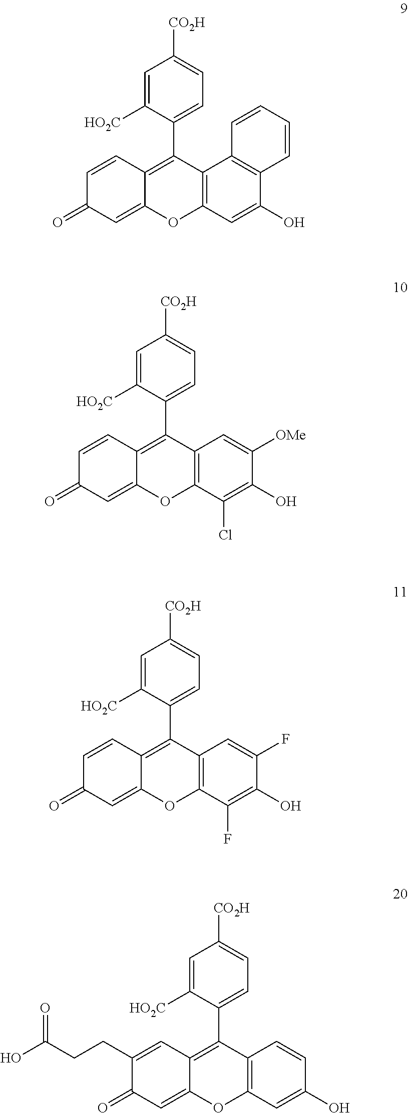 [SMILES: O=C(O)c1ccc(-c2c3ccc(=O)cc-3oc3cc(O)c4ccccc4c23)c(C(=O)O)c1 ; COc1cc2c(-c3ccc(C(=O)O)cc3C(=O)O)c3ccc(=O)cc-3oc2c(Cl)c1O ; O=C(O)c1ccc(-c2c3ccc(=O)cc-3oc3c(F)c(O)c(F)cc23)c(C(=O)O)c1 ; O=C(O)CCc1cc2c(-c3ccc(C(=O)O)cc3C(=O)O)c3ccc(O)cc3oc-2cc1=O]

Other interesting compounds B derived from compound 6 are:

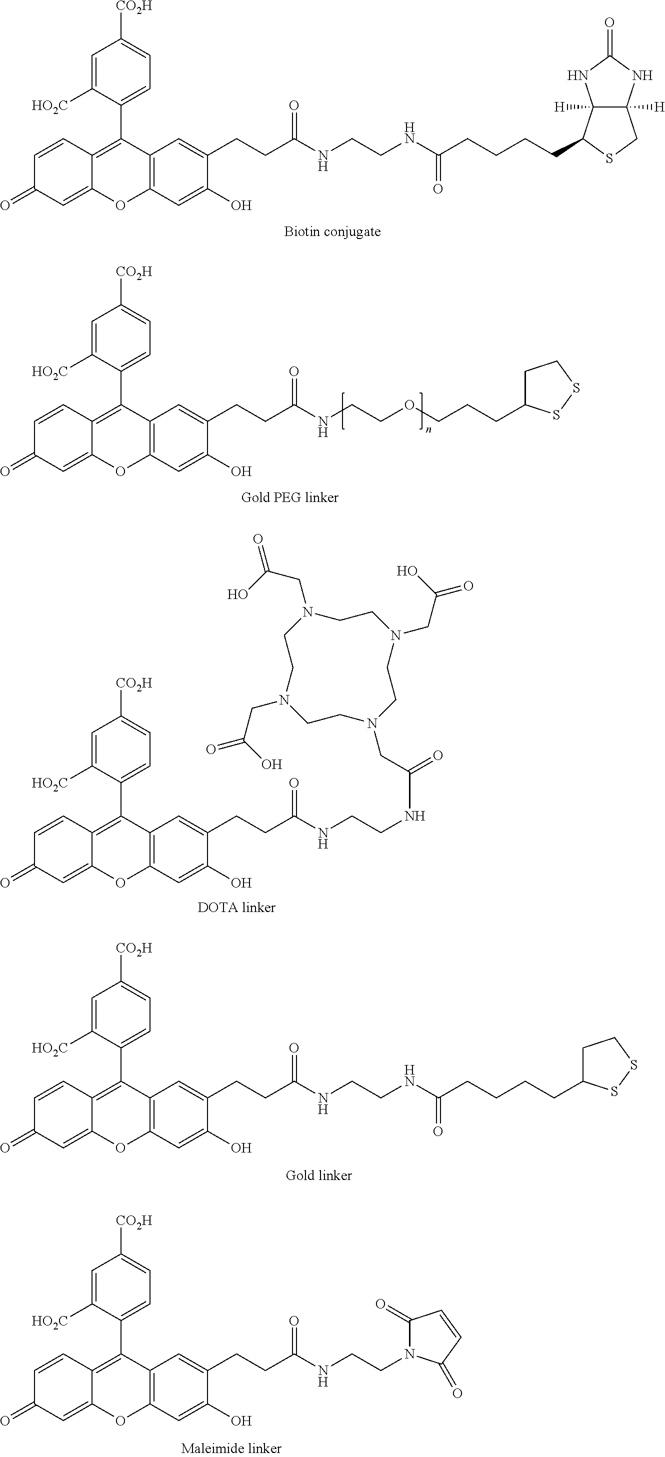

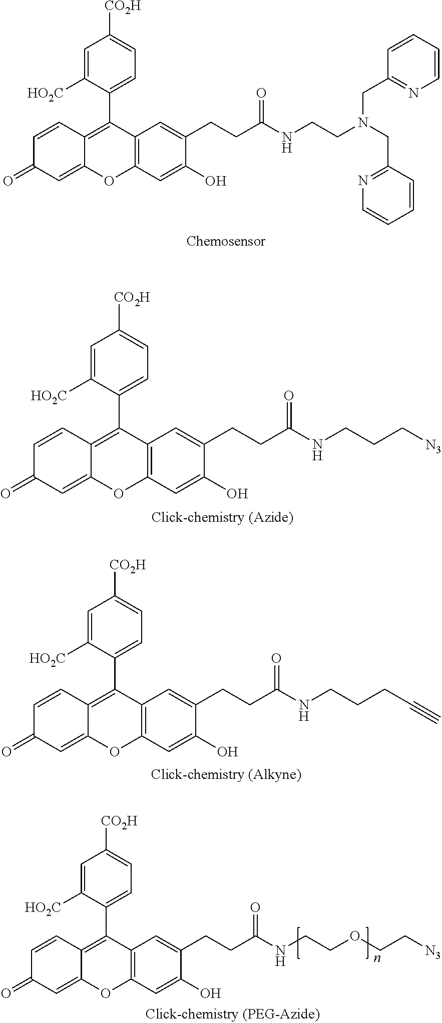

Some of the most interesting compounds C derived from compound 13 are:

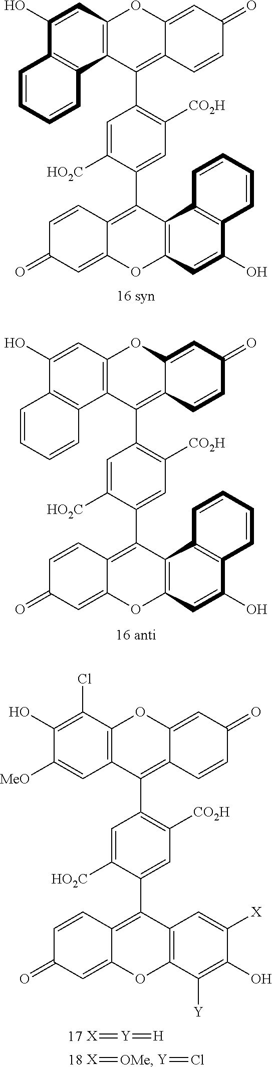

### Novel Carboxy-Fluoresceins

It is believed that some of the carboxy-fluoresceins of formula B and of formula C which are obtainable from the method described further above represent hitherto unknown chemical entities.

Hence, the invention further provides novel compounds of formula B*

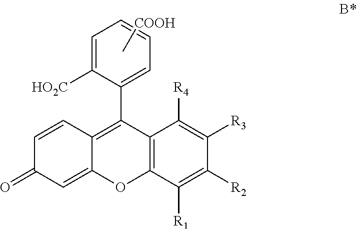

wherein R₁, R₂, R₃ and R₄ are independently selected from hydrogen; halogen; hydroxyl; nitro; cyano; mercapto; —O—C₁₋₃-alkyl; —S—C₁₋₃-alkyl; cyclopropyl; —C₁₋₃-alkyl; —C₂₋₃-alkenyl; or —C₂₋₃-alkynyl; which —O—C₁₋₃-alkyl, —S—C₁₋₃-alkyl, cyclopropyl, —C₁₋₃-alkyl, —C₂₋₃-alkenyl or —C₂₋₃-alkynyl is optionally substituted with at least one substituent selected from halogen, hydroxyl, nitro, cyano and mercapto; with the additional option that any of the substituent pairs, R₁/R₂, R₂/R₃ and R₃/R₄ together with the intervening atoms may form an optionally substituted aromatic ring or ring system, with the proviso that when R₁=R₃=R₄=hydrogen, then R₂ is different from hydroxyl.

Suitably, in compounds B*, R₁, R₂, R₃ and R₄ are not all hydrogen. Preferably, in compounds B*, R₂ is hydroxyl (—OH). Suitably, R₁ is halogen, most preferably F or Cl. R₃ may be halogen, preferably F or CI, or —O—C₁₋₃-alkyl, such as —OCH₃. Suitably, R₃/R₄ together with the intervening atoms form an optionally substituted aromatic ring system. Preferred compounds B* of the invention are compounds 9, 10 and 11 of Scheme 1.

Also, the invention further provides novel compounds of formula C*

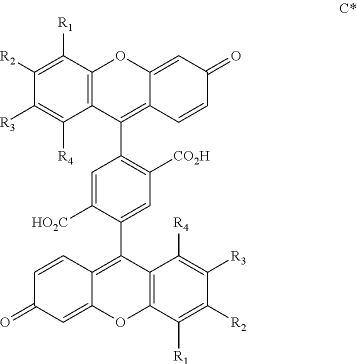

wherein R₁, R₂, R₃ and R₄ are independently selected from hydrogen; halogen; hydroxyl; nitro; cyano; mercapto; —O—C₁₋₃-alkyl; —S—C₁₋₃-alkyl; cyclopropyl; —C₁₋₃-alkyl; —C₂₋₃-alkenyl; or —C₂₋₃-alkynyl; which —O—C₁₋₃-alkyl, —S—C₁₋₃-alkyl, cyclopropyl, —C₁₋₃-alkyl, —C₂₋₃-alkenyl or —C₂₋₃-alkynyl is optionally substituted with at least one substituent selected from halogen, hydroxyl, nitro, cyano and mercapto; with the additional option that any of the substituent pairs, R₁/R₂, R₂/R₃ and R₃/R₄ together with the intervening atoms may form an optionally substituted aromatic ring or ring system, with the proviso that when R₁=R₃=R₄=hydrogen, then R₂ is different from hydroxyl.

Suitably, in compounds C*, R₁, R₂, R₃ and R₄ are not all hydrogen. Preferably, in compounds B*, R₂ is hydroxyl (—OH). Suitably, R₁ is halogen, most preferably F or Cl. R₃ may be halogen, preferably F or CI, or —O—C₁₋₃-alkyl, such as —OCH₃. Suitably, R₃/R₄ together with the intervening atoms form an optionally substituted aromatic ring system. Preferred compounds C* of the invention are compounds 16 syn, 16 anti, 17 and 18 of Scheme 2.

Also, the invention further provides the novel compounds 5 and 6 of the formulae

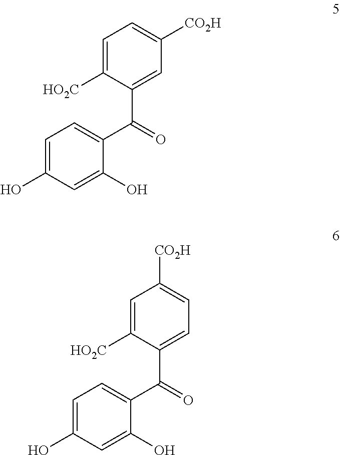 [SMILES: O=C(O)c1ccc(C(=O)O)c(C(=O)c2ccc(O)cc2O)c1 ; O=C(O)c1ccc(C(=O)c2ccc(O)cc2O)c(C(=O)O)c1]

### EXPERIMENTAL SECTION

### Schemes 1 and 2

Unless otherwise stated, all starting materials were obtained from commercial suppliers and used as received. Solvents were HPLC grade and were used as received. High resolution mass spectra (HR-MS) were measured on a Ultimate 3000 Dionex UHPLC, Bruker Maxis 3G QTOF ESI MS. Reverse phase analytical LCMS was run on a Waters Acquity Ultra Performance LCMS. NMR spectra were recorded using a Varian Mercury 300 MHz spectrometer or a Bruker 500 MHz spectrometer. Chemical shifts were measured in ppm and coupling constants in Hz, the field is indicated in each case. When DMSO-d₆ was used, the values were δ 2.50 for ¹H NMR and δ 39.43 for ¹³C NMR spectra. When D₂O added NaOD was used as solvent, the residual peak was used as internal reference at δ 4.79 for ¹H NMR spectrum. Melting points were measured with a Buch & Holm melting point apparatus and are uncorrected. TLC was performed on Merck aluminum sheets pre-coated with silica gel 60 F254. Gravity feed column chromatography was performed on Merck Kiselgel 60 (0.040-0.063 mm).

### 2-(2,4-Dihydroxybenzoyl)terephthalic acid (5) and 4-(2,4-dihydroxybenzoyl)isophthalic acid (6)

In a 250 mL conical flask equipped with a reflux condenser and a magnetic stirrebar were placed 1,3-dioxo-1,3-dihydroisobenzofuran-5-carboxylic acid (20 g, 0.104 mol) and resorcinol (23 g, 0.208 mol) in 100 mL methanesulfonic acid. The reaction mixture were stirred at 80° C. overnight, added to 500 mL ice water under stirring and filtered. The solid residue was refluxed in 200 mL EtOH, added H₂O until precipitation, cooled to room temperature, filtered and dried in vacuo yielding 36.5 g (93%) of crude 5(6)-Carboxy-fluorescein as an orange powder (only compound seen on LCMC). The crude compound was used without further purification. In a 500 mL conical flask equipped with a reflux condenser and a magnetic stirrebar were placed H₂O (200 g) and NaOH (200 g) were added under heat evolution. To the warm mixture were added crude 5(6)-Carboxy-fluorescein and the mixture was stirred at 80° C. overnight at which time the solution had become clear and almost colorless. The solution was added to 300 g ice and further cooled with ice. 12 M HCl were added slowly under stirring until a white compound precipitates (pH=1-2). The mixture was left at 5° C. overnight, filtered and dried in vacuo yielding the crude mixture of isomers (approximately a 1:1 ratio) as an off white solid. The mixture was fractional crystallized by dissolving the mixture in MeOH (100 mL) and subsequently adding H₂O (3 L). Small crystals starts forming on the surface of the solution overnight and the solution is left standing at RT for one week in an open Erlenmeyer flask. The crystals are collected and the mother liquor is extracted with diethyl ether. The ether phase was evaporated to dryness, and crystallized using the same procedure as before in MeOH—H₂O. The combined solid (benzophenone 6) was recrystallized 2-3 times, each time combining the mother liquor (containing mostly benzophenone 5), yielding 11.5 g (36%) of benzophenone 6. Isolation of benzophenone 5 was achieved by combining the dried ether phases and crystallizing them in H₂O 4-5 times yielding 8.3 g (26%).

### 2-(2,4-Dihydroxybenzoyl)terephthalic acid (5)

Mp: 271-274 (decompose); 1H NMR (400 MHz, DMSO) δ 13.54 (s, 2H), 12.01 (s, 1H), 10.75 (s, 1H), 8.16 (dd, J=8.1, 1.7 Hz, 1H), 8.09 (d, J=8.0 Hz, 1H), 7.85 (d, J=1.4 Hz, 1H), 6.99 (d, J=8.7 Hz, 1H), 6.34-6.29 (m, 2H); ¹³C NMR (101 MHz, DMSO) δ 199.41, 166.74, 166.48, 165.58, 164.72, 140.70, 135.16, 134.30, 133.81, 130.91, 130.84, 128.38, 113.61, 108.91, 103.02; MS (ESI⁺) m/z [M+H⁺] calcd for C₁₅H₁₁O₇⁺ 303.0. found 302.9. HR-MS (ESI): m/z [M+H⁺] calcd for C₁₅H₁₁O₇⁺ 303.0499 found 303.0503.

### 4-(2,4-Dihydroxybenzoyl)isophthalic acid (6)

Mp: 265-267 (decompose); 1H NMR (500 MHz, DMSO) δ 13.48 (s, 1H), 12.00 (s, 1H), 10.76 (s, 1H), 8.51 (d, J=1.7 Hz, 1H), 8.23 (dd, 3=7.9, 1.7 Hz, 1H), 7.56 (d, 3=7.9 Hz, 1H), 6.96 (d, 3=8.8 Hz, 1H), 6.45-6.17 (m, 1H); ¹³C NMR (126 MHz, DMSO) δ 199.20, 166.03, 165.91, 165.12, 164.13, 143.83, 134.58, 132.84, 131.79, 130.68, 129.72, 127.95, 113.10, 108.43, 102.49. MS (ESI⁺) m/z [M+H⁺] calcd for C₁₅H₁₁O₇⁺ 303.0. found 302.9. HR-MS (ESI): m/z [M+H⁺] calcd for C₁₅H₁₁O₇⁺ 303.0499 found 303.0503.

### General Method. 2-(6-hydroxy-3-oxo-3H-xanthen-9-yl)terephthalic acid (7)

In a 20 mL conical flask were placed 2-(2,4-dihydroxybenzoyl)terephthalic acid (5) (200 mg, 0.66 mmol), resorcinol (80 mg, 0.72 mmol) in 5 mL methanesulfonic acid. The reaction mixture were stirred at RT overnight, added to 50 mL ice water under stirring and filtered. The solid residue was dissolved in 2M NaOH (40 mL), precipitated with 2M HCl and filtered. The crude compound was re-precipitated first in EtOH/H₂O and followed by NaOH/HCl, filtered and dried in vacuo. Yield: 245 mg, 98%; Mp: >300° C.; ¹H NMR (400 MHz, D₂O(NaOD)) δ 8.04 (dd, J=8.0, 1.7 Hz, 1H), 7.83 (d, J=8.0 Hz, 1H), 7.75 (d, J=1.3 Hz, 1H), 7.21-7.13 (m, 2H), 6.64-6.54 (m, 4H); ¹³C NMR (101 MHz, D₂O(NaOD)) δ 180.61, 174.86, 174.35, 158.85, 158.65, 141.76, 137.05, 131.55, 131.30, 130.24, 129.72, 128.10, 122.87, 112.50, 103.52; MS (ESI⁺) m/z [M+H⁺] calcd for C₂₁H₁₃O₇⁺ 377.1. found 377.0; HR-MS (ESI): m/z [M+H⁺] calcd for C₂₁H₁₃O₇⁺ 377.0655 found 377.0676.

### 4-(6-hydroxy-3-oxo-3H-xanthen-9-yl)isophthalic acid (8)

The compound was prepared as in the case of compound 7, starting from 4-(2,4-Dihydroxybenzoyl)isophthalic acid (6) (0.5 g, 1.65 mmol) and resorcinol (0.2 g, 1.82 mmol) and 10 mL methanesulfonic acid. Yield: 602 mg, 96%; Mp: >300° C.; ¹H NMR (400 MHz, D₂O(NaOD)) δ 8.21 (d, J=1.6 Hz, 1H), 8.00 (dd, J=7.9, 1.7 Hz, 1H), 7.24 (d, J=7.9 Hz, 1H), 7.13 (d, J=9.2 Hz, 2H), 6.58 (dd, J=9.2 Hz, J=2.2 Hz, 2H), 6.54 (d, J=2.2 Hz, 2H); ¹³C NMR (101 MHz, D₂O(NaOD))) δ 180.59, 174.74, 174.49, 158.67, 139.59, 137.36, 134.08, 131.40, 130.05, 129.45, 128.43, 122.88, 112.19, 103.58; MS (ESI⁺) m/z [M+H⁺] calcd for C₂H₁₃O₇⁺ 377.1. found 377.1; HR-MS (ESI): m/z [M+H⁺] calcd for C₂₁H₁₃O₇⁺ 377.0655 found 377.0667.

### 4-(5-hydroxy-9-oxo-9H-benzo[a]xanthen-12-yl)isophthalic acid (9)

The compound was prepared as in the case of compound 7, starting from 4-(2,4-dihydroxybenzoyl)isophthalic acid (6) (500 mg, 1.65 mmol), naphthalene-1,3-diol (500 mg, 3.12 mmol) and 10 mL methanesulfonic acid. Yield: 557 mg, 79%; Mp: >300° C.; ¹H NMR (400 MHz, D₂O(NaOD)) δ 8.22 (d, J=1.5 Hz, 1H), 8.10 (dd, J=8.1, 1.3 Hz, 1H), 7.78 (dd, J=7.9, 1.8 Hz, 1H), 7.37-7.28 (m, 1H), 7.08 (ddd, J=8.6, 7.1, 1.5 Hz, 1H), 6.88 (d, J=8.5 Hz, 1H), 6.81-6.74 (m, 1H), 6.71 (d, J=7.9 Hz, 1H), 6.47 (dd, J=7.5, 2.2 Hz, 2H), 6.33 (s, 1H); ¹³C NMR (101 MHz, D₂O(NaOD)) δ 180.42, 176.93, 174.50, 173.99, 162.98, 155.72, 155.05, 138.58, 138.12, 137.14, 131.55, 130.83, 130.25, 129.84, 129.51, 129.43, 128.57, 126.71, 126.26, 124.66, 121.20, 111.73, 109.80, 103.14, 101.30; MS (ESI⁺) m/z [M+H⁺] calcd for C₂₅H₁₅O₇⁺ 427.1 found 427.1; HR-MS (ESI): m/z [M+H⁺] calcd for C₂₅H₁₅O₇⁺ 427.0812 found 427.0835.

### 4-(5-chloro-6-hydroxy-7-methoxy-3-oxo-3H-xanthen-9-yl)isophthalic acid (10)

The compound was prepared as in the case of compound 7, starting from 4-(2,4-dihydroxybenzoyl)isophthalic acid (6) (300 mg, 0.99 mmol), 2-chloro-4-methoxybenzene-1,3-diol (200 mg, 1.15 mmol) and 10 mL methanesulfonic acid. The crude compound was purification by silica gel dry column vacuum chromatography was performed by dissolving the crude compound in MeOH and 2 drops of 12 M NaOH(aq), evaporation on celite in vacuo, using 2% AcOH in CH₂Cl₂/MeOH with 5% increments. The compound was re-precipitated in NaOH/HCl, filtered and dried in vacuo. Yield: 265 mg, 60%; Mp: >300° C.; ¹H NMR (400 MHz, D₂O(NaOD)) δ 8.21 (d, J=1.6 Hz, 1H), 8.04 (dd, J=7.9, 1.7 Hz, 1H), 7.27 (d, J=7.9 Hz, 1H), 7.05 (d, J=9.2 Hz, 1H), 6.65 (d, J=2.2 Hz, 1H), 6.59 (dd, J=9.2, 2.2 Hz, 1H), 6.31 (s, 1H), 3.57 (s, 3H); ¹³C NMR (101 MHz, D₂O(NaOD)) δ 178.91, 174.64, 174.41, 168.26, 157.27, 156.08, 150.98, 150.93, 139.71, 137.41, 134.05, 130.38, 130.27, 129.54, 128.57, 122.81, 111.86, 110.88, 107.82, 103.60, 55.34; MS (ESI⁺) m/z [M+H⁺] calcd for C₂₂H₁₄ClO₈⁺ 441.0. found 441.0; HR-MS (ESI): m/z [M+H⁺] calcd for C₂₂H₁₄ClO₈⁺ 441.0372. found 441.0377.

### 4-(5,7-difluoro-6-hydroxy-3-oxo-3H-xanthen-9-yl)isophthalic acid (11)

The compound was prepared as in the case of compound 7, starting from 4-(2,4-dihydroxybenzoyl)isophthalic acid (6) (600 mg, 1.98 mmol), 2,4-difluorobenzene-1,3-diol (440 mg, 3.9 mmol) and 10 mL methanesulfonic acid. The crude compound was purification by silica gel dry column vacuum chromatography was performed by dissolving the crude compound in MeOH and 2 drops of 12 M NaOH(aq), evaporation on celite in vacuo, using 2% AcOH in CH₂Cl₂/MeOH with 5% increments. The compound was re-precipitated in NaOH/HCl, filtered and dried in vacuo. Yield: 237 mg, 28%; Mp=259-265° C. (decompose); ¹H NMR (400 MHz, D₂O(NaOD)) δ 8.16 (d, J=1.4 Hz, 1H), 7.97 (d, J=7.9 Hz, 1H), 7.25 (d, J=7.9 Hz, 1H), 7.09 (d, J=9.4 Hz, 1H), 6.74 (dd, J=11.6, 1.3 Hz, 1H), 6.56-6.49 (m, 2H); ¹³C NMR (101 MHz, D₂O(NaOD)) δ 182.02, 174.44, 174.43, 158.49, 139.54, 137.67, 133.65, 131.55, 130.02, 129.64, 128.65, 124.10, 113.14, 108.53, 108.32, 103.71; ¹⁹F NMR (282 MHz, D₂O(NaOD)) δ 127, 154; MS (ESI⁺) m/z [M+H⁺] calcd for C₂₁H₁₁F₂O₂⁺ 413.0. found 413.0; HR-MS (ESI): m/z [M+H⁺] calcd for C₂₁H₁₁F₂O₂⁺ 413.0467 found 413.0478.

### 2,5-bis(2,4-dihydroxybenzoyl)terephthalic acid (13)

In a 100 mL conical flask equipped with a reflux condenser and a magnetic stirrebar were placed benzo[1,2-c:4,5-c′]difuran-1,3,5,7-tetraone (12) (5 g, 22.9 mmol) and resorcinol (10 g, 91.6 mmol) in 60 mL methanesulfonic acid. The reaction mixture were stirred at 80° C. overnight, added to 500 mL ice water under stirring and filtered. The solid residue was refluxed in 200 mL EtOH, cooled to room temperature, filtered and dried in vacuo yielding 12 g of crude 2,5-bis(3,6-dihydroxy-9-(methoxysulfonyl)-9H-xanthen-9-yl)terephthalic acid as an orange powder. The crude compound was used without further purification. In a 250 mL conical flask equipped with a reflux condenser and a magnetic stirrebar were placed H₂O (100 g) and NaOH (100 g) were added under heat evolution. To the warm mixture were added crude 2,5-bis(3,6-dihydroxy-9-(methoxysulfonyl)-9H-xanthen-9-yl)terephthalic acid and the mixture were stirred at 80° C. overnight at which time the solution had become clear and almost colorless. The solution was added to 200 g ice and further cooled with ice. 12M HCL were added slowly under stirring until a white compound precipitates (pH=1-2). The mixture was left overnight at 5° C., filtered and the solid was dried in vacuo yielding a white powder. Yield: 5.5 g, 55%; Mp: 283-286 (decompose); ¹H NMR (500 MHz, DMSO) δ 13.70 (s, 2H), 11.92 (s, 2H), 10.91 (s, 2H), 7.90 (s, 2H), 7.17 (d, J=8.7 Hz, 2H), 6.49-6.15 (m, 4H); ¹³C NMR (101 MHz, DMSO)) δ 197.95, 165.61, 165.34, 164.12, 140.97, 134.82, 132.62, 128.79, 113.12, 108.55, 102.54; MS (ESI⁺) m/z [M+H⁺] calcd for C₂₅H₁₅O₁₀⁺ 439.1. found 439.0; HR-MS (ESI): m/z [M+H⁺] calcd for C₂₅H₁₅O₁₀⁺ 439.0659 found 439.0658.

### General Method. Mixture of 2-(5,7-difluoro-6-hydroxy-3-oxo-3H-xanthen-9-yl)-5-(6-hydroxy-3-oxo-3H-xanthen-9-yl)terephthalic acid (14) and 2,5-bis(5,7-difluoro-6-hydroxy-3-oxo-3H-xanthen-9-yl)terephthalic acid (15)

A mixture of 2,5-bis(2,4-dihydroxybenzoyl)terephthalic acid (13) (100 mg, 0.23 mmol) and 2,4-difluorobenzene-1,3-diol (100 mg, 0.7 mmol) in methanesulfonic acid (20 mL) was placed in a 50 mL conical flask equipped with a magnetic stirrebar and the mixture was heated to 50° C. overnight. 2,4-difluorobenzene-1,3-diol (100 mg, 0.7 mmol) was added and the reaction was stirred for 2 days at 50° C., added to 50 mL ice water under stirring and filtered. The solid residue was dissolved in 2M NaOH (40 mL), precipitated with 2M HCl and filtered. The crude compound was purified by dry column vacuum chromatography (5% AcOH in Toluene to 40% EtOH in 5% AcOH in Toluene with 4% increments) giving a mixture of compound 14 ((ESI⁺) m/z [M+H⁺] calcd for C₃₄H₁₂F₂O₁₀⁺ 623.1 found 623.0) and compound 15 ((ESI⁺) m/z [M+H⁺] calcd for C₃₄H₁₅F₄O₁₀⁺ 659.1 found 659.1.

### 2,5-bis(5-hydroxy-9-oxo-9H-benzo[a]xanthen-12-yl)terephthalic acid (16 anti) and (16 syn)

The compounds were prepared as in the case of compound 14, starting from 2,5-bis(2,4-dihydroxybenzoyl)terephthalic acid (13) (500 mg, 1.14 mmol), naphthalene-1,3-diol (500 mg, 3.12 mmol) and 10 mL methanesulfonic acid. The reaction mixture was stirred at 80° C. for 2 hours. Yield: 665 mg, 85%; Mp: >300° C.; ¹³C NMR (101 MHz, D₂O(NaOD)) δ 13C NMR (101 MHz, D20) δ 180.79, 180.65, 177.12, 176.96, 172.71, 172.64, 163.54, 163.48, 156.09, 156.01, 154.54, 154.39, 140.74, 140.70, 137.34, 137.23, 131.87, 131.81, 130.63, 130.60, 130.07, 129.85, 129.50, 129.40, 129.36, 127.19, 127.01, 126.54, 126.48, 124.97, 124.92, 121.58, 121.53, 111.94, 111.71, 110.31, 110.27, 103.29, 103.22, 101.38, 101.35, 99.99.; MS (ESI⁺) m/z [M+H⁺] calcd for C₄₂H₂₃O₁₀⁺ 687.1 found 687.1. HR-MS (ESI-TOF): m/z calcd for C₄₂H₂₃O₁₀⁺ 687.1286 found 687.1289.

### 2-(5-chloro-6-hydroxy-7-methoxy-3-oxo-3H-xanthen-9-yl)-5-(6-hydroxy-3-oxo-3H-xanthen-9-yl)terephthalic acid (17) and 2,5-bis(5-chloro-6-hydroxy-7-methoxy-3-oxo-3H-xanthen-9-yl)terephthalic acid (18)

The compounds were prepared as in the case of compound 18, starting from 2,5-bis(2,4-dihydroxybenzoyl)terephthalic acid (14) (100 mg, 0.23 mmol), 2-chloro-4-methoxybenzene-1,3-diol (90 mg, 0.52 mmol) and 3 mL methanesulfonic acid. Compound 17. Yield: 34 mg, 23%; Mp: >300° C.; ¹H NMR (400 MHz, D₂O(NaOD)) δ 8.04 (s, 1H), 7.83 (s, 1H), 7.35 (dd, J=19.6, 9.2 Hz, 2H), 7.26 (d, J=9.2 Hz, 1H), 6.80 (d, J=2.2 Hz, 1H), 6.72-6.70 (m, 1H), 6.70-6.68 (m, 2H), 6.66 (d, J=2.3 Hz, 1H), 6.65-6.64 (m, 2H), 6.54 (s, 1H), 3.74 (s, 3H); ¹³C NMR (101 MHz, D₂O(NaOD)) δ 180.75, 179.11, 173.68, 173.51, 168.39, 158.90, 158.86, 157.84, 157.52, 155.45, 151.12, 151.08, 140.55, 140.25, 132.90, 132.80, 131.44, 130.51, 130.33, 130.09, 123.18, 123.13, 122.99, 112.45, 112.42, 112.07, 111.26, 107.90, 103.95, 103.67, 103.63, 55.57; MS (ESI⁺) m/z [M+H⁺] calcd for C₃₅H₂₀ClO₁₁⁺ 651.9 found 651.0. Compound 18. Yield: 34 mg, 21%; Mp: >300° C.; ¹H NMR (400 MHz, D₂O(NaOD)) δ 7.96 (s, 1H), 7.34 (d, J=9.2 Hz, 1H), 6.84 (d, J=2.2 Hz, 1H), 6.72 (dd, J=9.2, 2.2 Hz, 1H), 6.57 (s, 1H), 3.70 (s, 3H); ¹³C NMR (101 MHz, D₂O(NaOD)) δ 179.10, 179.05, 173.58, 173.46, 168.33, 168.27, 157.46, 157.43, 155.43, 155.37, 151.03, 150.91, 140.55, 140.52, 132.90, 132.75, 130.53, 122.91, 111.97, 111.92, 111.20, 107.83, 107.78, 103.70, 55.54, 55.00; MS (ESI⁺) m/z [M+H⁺] calcd for C₃₆H₂₁Cl₂O₁₂⁺ 716.4 found 715.0.

### Scheme 3

Unless otherwise stated, all starting materials were obtained from commercial suppliers and used as received. Solvents were HPLC grade and were used as received. High resolution mass spectra (HR-MS) were measured on a Ultimate 3000 Dionex UHPLC, Bruker Maxis 3G QTOF ESI MS. Reverse phase analytical LCMS was run on a Water UPLC-MS. NMR spectra were recorded using a Varian Mercury 300 MHz spectrometer or a Bruker 500 MHz spectrometer. Chemical shifts were measured in ppm and coupling constants in Hz, the field is indicated in each case. When DMSO-d6 was used, the values were δ 2.50 for 1H NMR and δ 39.43 for 13C NMR spectra. When D20 added NaOD was used as solvent, the residual peak was used as internal reference at δ 4.79 for 1H NMR spectrum. Melting points were measured with a Buch & Holm melting point apparatus and are uncorrected. TLC was performed on Merck aluminum sheets pre-coated with silica gel 60 F254. Gravity feed column chromatography was performed on Merck Kieselgel 60 (0.040-0.063 mm).

### General Procedure for the Syntheses of 5-carboxy-SNAFLs

4-(2,4-Dihydroxybenzoyl)isophthalic acid (500 mg, 1.65 mmol) and the appropriate dihydroxynaphthalene (275 mg, 1.75 mmol) were dissolved in TFA (5 mL) and methanesulfonic acid (5 mL). The reaction mixture was stirred at room temperature overnight. The reaction was quenched by adding H₂O (25 mL) and the resulting dark purple precipitate was collected by centrifugation. After decantation the sediment was dissolved in NaOH(aq) (2 M, 15 mL) and precipitated with HCl(aq) (2 M, 20 mL). After decantation the sediment was washed with H₂O (2×35 mL) and re-precipitated by dissolving in EtOH (10 mL) and precipitated with H₂O (ad H₂O until precipitation). After decantation and washing with H₂O (2×35 mL) the crude compound was dried in vacuo yielding a dark purple powder. Further purification by silica gel dry column vacuum chromatography was performed by dissolving the crude compound in MeOH and 2 drops of 12 M NaOH(aq), evaporation on celite in vacuo, using 2% AcOH in CH₂Cl₂/MeOH with 5% increments was done if required.

### 5-Carboxy-SNAFL-282

Starting from 1,6-dihydroxynaphthalene (275 mg, 1.75 mmol) and 4-(2,4-dihydroxybenzoyl)isophthalic acid (500 mg, 1.65 mmol). Crude yield: 574 mg, 79%; Mp: 267-271° C.; ¹H NMR (400 MHz, acetone-d6) δ 8.58 (dd, J=1.4, 0.6 Hz, 1H), 8.48 (d, J=9.1 Hz, 1H), 8.41 (dd, J=8.0, 1.4 Hz, 1H), 7.46 (dd, J=8.0, 0.6 Hz, 1H), 7.41 (d, J=8.7 Hz, 1H), 7.34 (dd, J=9.1, 2.4 Hz, 1H), 7.26 (d, J=2.4 Hz, 1H), 7.04 (d, J=2.4 Hz, 1H), 6.82 (d, J=8.7 Hz, 1H), 6.78 (d, J=8.8 Hz, 1H), 6.72 (dd, J=8.8, 2.4 Hz, 1H); ¹³C NMR (101 MHz, acetone-d6) δ 169.68, 167.35, 161.33, 159.07, 158.97, 153.82, 148.94, 138.33, 137.94, 134.74, 131.26, 129.17, 127.73, 126.41, 126.08, 125.66, 124.09, 120.51, 119.82, 114.75, 111.75, 111.46, 111.25, 104.48, 85.13; MS (ESI⁺) m/z [M+H⁺] calcd for C₂₅H₁₄O₇⁺ 427.1. found 427.1. HR-MS (ESI): m/z [M+H⁺] calcd for C₂₅H₁₄O₇⁺ 427.0812. found 427.0835.

### 5-Carboxy-SNAFL-285

Starting from 2,6-dihydroxynaphthalene (275 mg, 1.75 mmol) and 4-(2,4-dihydroxybenzoyl)isophthalic acid (500 mg, 1.65 mmol). Crude yield: 580 mg, 80%; Mp: >300° C.; ¹H NMR (400 MHz, acetone-d6) δ 9.03 (s, ½H), 8.67 (s, 1H), 8.64 (s, ½H), 8.33 (dd, J=8.0, 1.5 Hz, 1H), 7.93 (d, J=9.0 Hz, 1H), 7.46 (d, J=9.0 Hz, 1H), 7.36 (d, J=8.0 Hz, 1H), 7.27 (d, J=2.7 Hz, 1H), 7.03 (d, J=9.3 Hz, 1H), 6.91 (dd, J=9.3, 2.7 Hz, 1H), 6.78 (d, J=1.9 Hz, 1H), 6.67 (d, J=2.3 Hz, 2H); ¹³C NMR (101 MHz, acetone-d6) δ 170.16, 167.09, 160.88, 160.83, 155.78, 152.18, 150.69, 138.30, 135.14, 134.19, 133.46, 130.54, 129.16, 128.13, 126.86, 126.82, 125.77, 120.81, 120.26, 114.70, 113.19, 112.48, 109.95, 103.71, 85.27.; MS (ESI⁺) m/z [M+H⁺] calcd for C₂₅H₁₄O₇⁺ 427.1. found 427.1. HR-MS (ESI): m/z [M+H⁺] calcd for C₂₅H₁₄O₇⁺ 427.0812 found 427.0833.

### 5-Carboxy-SNAFL-287

Starting from 1,8-dihydroxynaphthalene (50 mg, 312 μmol) and 4-(2,4-dihydroxybenzoyl)isophthalic acid (100 mg, 331 μmol). Purification by chromatography necessary. Yield: 78 mg, 59%; Mp: 235-239° C.; ¹H NMR (400 MHz, acetone-d6) δ 8.59 (dd, J=1.5, 0.7 Hz, 1H), 8.42 (dd, J=8.0, 1.5 Hz, 1H), 7.61-7.47 (m, 3H), 7.43 (dd, J=8.1, 0.7 Hz, 1H), 7.15 (d, J=2.4 Hz, 1H), 7.08 (dd, J=7.7, 1.1 Hz, 1H), 6.85 (m, 2H), 6.78 (dd, J=8.7, 2.4 Hz, 1H); ¹³C NMR (101 MHz, acetone-d6) δ 169.56, 167.30, 161.40, 158.75, 156.13, 152.76, 149.97, 138.68, 138.07, 134.89, 131.14, 130.97, 128.95, 127.85, 126.45, 126.28, 125.68, 121.23, 115.45, 115.18, 114.41, 113.80, 111.36, 104.70, 84.32; MS (ESI⁺) m/z [M+H⁺] calcd for C₂₅H₁₄O₇⁺ 427.1. found 427.1. HR-MS (ESI): m/z [M+H⁺] calcd for C₂₅H₁₄O₇⁺ 427.0812 found 427.0832.

### 5-Carboxy-SNAFL-289

Starting from 1,4-dihydroxynaphthalene (275 mg, 1.75 mmol) and 4-(2,4-dihydroxybenzoyl)isophthalic acid (500 mg, 1.65 mmol). Crude yield: 525 mg, 74%; Mp: 268-272° C.; ¹H NMR (400 MHz, acetone-d6) δ 8.58 (s, 1H), 8.55 (d, J=8.0 Hz, 1H), 8.42 (dd, J=8.0, 1.3 Hz, 1H), 8.25 (d, J=7.8 Hz, 1H), 7.75 (ddd, J=8.2, 7.0, 1.3 Hz, 1H), 7.68 (ddd, J=8.2, 7.0, 1.3 Hz, 1H), 7.47 (d, J=8.0 Hz, 1H), 7.03 (d, J=2.4 Hz, 1H), 6.84 (d, J=8.7 Hz, 1H), 6.71 (dd, J=8.7, 2.4 Hz, 1H), 6.15 (s, 1H); ¹³C NMR (101 MHz, acetone-d6) δ 169.69, 167.45, 161.30, 158.91, 154.02, 150.78, 142.18, 137.99, 135.06, 131.27, 129.08, 128.98, 128.89, 128.05, 127.57, 126.59, 126.47, 124.13, 123.61, 114.56, 114.05, 111.04, 105.36, 104.37, 85.30.; MS (ESI⁺) m/z [M+H⁺] calcd for C₂₅H₁₄O₇⁺ 427.1. found 427.1. HR-MS (ESI): m/z [M+H⁺] calcd for C₂₅H₁₄O₇⁺ 427.0812 found 427.0835.

### 5-Carboxy-SNAFL-293

Starting from 2,3-dihydroxynaphthalene (54 mg, 331 μmol) and 4-(2,4-dihydroxybenzoyl)isophthalic acid (100 mg, 331 μmol). Purification by chromatography necessary. Yield: 41 mg, 29%; Mp: 205-208° C.; ¹H NMR (400 MHz, Acetone) δ 9.15 (s, 1H), 9.10 (s, 1H), 8.68 (d, J=0.7 Hz, 5H), 8.34 (dd, 3=8.0, 1.5 Hz, 1H), 7.76 (d, J=8.1 Hz, 1H), 7.52 (s, 6H), 7.42 (dd, J=8.0, 0.6 Hz, 1H), 7.28 (ddd, J=8.0, 5.9, 2.0 Hz, 6H), 7.11-6.98 (m, 2H), 6.97-6.91 (m, 1H), 6.71 (s, 1H), 6.70 (s, 2H); ¹³C NMR (100 MHz, Acetone) δ 9.15, 9.10, 8.68, 8.68, 8.68, 8.68, 8.35, 8.34, 8.33, 8.32, 7.77, 7.75, 7.52, 7.43, 7.43, 7.41, 7.41, 7.30, 7.30, 7.29, 7.28, 7.28, 7.27, 7.26, 7.09, 7.08, 7.07, 7.06, 7.05, 7.05, 7.03, 6.95, 6.94, 6.94, 6.71, 6.70; MS (ESI⁺) m/z [M+H⁺] calcd for C₂₅H₁₄O₇⁺ 427.4. found 427.1. HR-MS (ESI): m/z [M+H⁺] calcd for C₂₅H₁₄O₇⁺ 427.0812 found 427.0825.

### 5-Carboxy-SNAFL-294

Starting from 1,5-dihydroxynaphthalene (54 mg, 331 μmol) and 4-(2,4-dihydroxybenzoyl)isophthalic acid (100 mg, 331 μmol). Crude yield: 105 mg, 75%; Mp: 243-245° C.; ¹H NMR (400 MHz, acetone-d6) δ 8.61 (dd, J=1.5, 0.6 Hz, 1H), 8.42 (dd, J=8.0, 1.5 Hz, 1H), 8.09 (d, J=8.4 Hz, 1H), 7.94 (dd, J=8.9, 0.6 Hz, 1H), 7.59-7.51 (m, 1H), 7.49 (dd, J=8.0, 0.6 Hz, 1H), 7.12 (dd, J=7.6, 0.7 Hz, 1H), 7.07 (d, J=2.4 Hz, 1H), 6.87 (d, J=8.7 Hz, 1H), 6.85 (d, J=8.9 Hz, 1H), 6.75 (dd, J=8.7, 2.4 Hz, 1H); ¹³C NMR (101 MHz, acetone-d6) δ 168.70, 166.27, 160.60, 157.99, 154.09, 152.97, 147.65, 137.06, 133.53, 130.36, 128.40, 128.19, 126.96, 126.45, 126.04, 125.63, 123.18, 118.92, 114.03, 113.93, 113.57, 111.46, 110.72, 103.59, 39.70; MS (ESI⁺) m/z [M+H⁺] calcd for C₂₅H₁₄O₇⁺ 427.1. found 427.1. HR-MS (ESI): m/z [M+H⁺] calcd for C₂₅H₁₄O₇⁺ 427.0812 found 427.0833.

The following are aspects of the invention

Aspect 1. A method for the preparation and isolating of compound 6 and, optionally, of compound 5

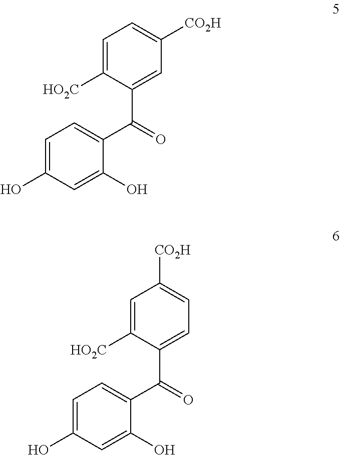 [SMILES: O=C(O)c1ccc(C(=O)O)c(C(=O)c2ccc(O)cc2O)c1 ; O=C(O)c1ccc(C(=O)c2ccc(O)cc2O)c(C(=O)O)c1]

said method comprising the steps of:
(i) providing a condensation product mixture, being the result of a condensation reaction between trimellitic anhydride and resorcinol mediated by acid;
(ii) hydrolysing said condensation product mixture with a strong aqueous base at pH at least 11;
(iii) acidifying the reaction mixture of step (ii) so as to isolate a mixture of compound 5 and compound 6;
(iv) dissolving the mixture of compound 5 and compound 6 in methanol and adding water so as to precipitate compound 6;
(v) extracting the mother liquor with an organic solvent so as to isolate compound 5 and any remaining compound 6, and removing the organic solvent so as to obtain a dried extract;
(vi) optionally repeating steps (iv) and (v) in one or more additional cycles using the dried extract obtained in step (v);
(vii) optionally dissolving the dried extract obtained in step (v) in refluxing H₂O and precipitating compound 5.

Aspect 2. A method for the preparation and isolation of compound 13

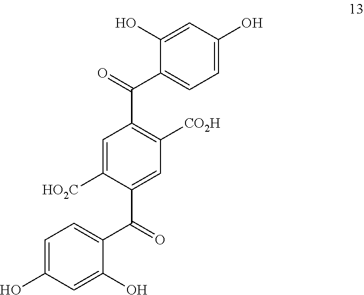 [SMILES: O=C(O)c1cc(C(=O)c2ccc(O)cc2O)c(C(=O)O)cc1C(=O)c1ccc(O)cc1O]

said method comprising the steps of:
(i) providing a condensation product, being the result of a condensation reaction between pyromellitic dianhydride and resorcinol mediated by acid;
(ii) hydrolysing said condensation product with a strong aqueous base at pH of at least 11;
(iii) acidifying the reaction mixture of step (ii) so as to isolate compound 13.

Aspect 3. The method according to any one of aspects 1-2, wherein hydrolysis steps (step ii.) are carried out at a pH of 12-14, preferably using a 1:1 weight ratio mixture of NaOH and H₂O.

Aspect 4. The method according to any one of aspects 1-3, wherein the acidification steps (step iii) are carried out using 12 M HCl.

Aspect 5. The method according to any one of aspects 1, 3 or 4, wherein, in step vi, steps (iv) and (v) are repeated in 2-3 additional cycles.

Aspect 6. The method according to any one of aspects 1, 3-5, wherein compound 5 or compound 6 is subsequently reacted with a compound of the formula A

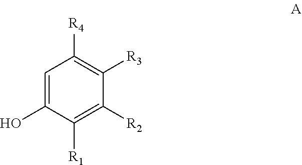

wherein R₁, R₂, R₃ and R₄ are independently selected from hydrogen; halogen; hydroxyl; nitro; cyano; mercapto; —O—C₁₋₃-alkyl; —S—C₁₋₃-alkyl; cyclopropyl; —C₁₋₃-alkyl; —C₂₋₃-alkenyl; or —C₂₋₃-alkynyl; which —O—C₁₋₃-alkyl, —S—C₁₋₃-alkyl, cyclopropyl, —C₁₋₃-alkyl, —C₂₋₃-alkenyl or —C₂₋₃-alkynyl is optionally substituted with at least one substituent selected from halogen, hydroxyl, nitro, cyano and mercapto; with the additional option that any of the substituent pairs, R₁/R₂, R₂/R₃ and R₃/R₄ together with the intervening atoms may form an optionally substituted aromatic ring or ring system;

in the presence of a strong acid (e.g. methanesulfonic acid) so as to provide a compound of formula B

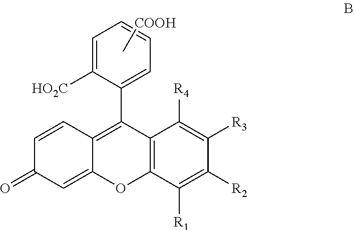

wherein R₁, R₂, R₃ and R₄ are as defined above.

Aspect 7. The method according to any one of aspects 2-5, wherein compound 13 is subsequently reacted with a compound of the formula A

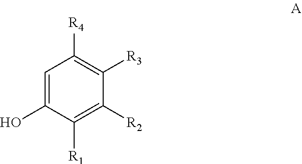

wherein R₁, R₂, R₃ and R₄ are independently selected from hydrogen; halogen; hydroxyl; nitro; cyano; mercapto; —O—C₁₋₃-alkyl; —S—C₁₋₃-alkyl; cyclopropyl; —C₁₋₃-alkyl; —C₂₋₃-alkenyl; or —C₂₋₃-alkynyl; which —O—C₁₋₃-alkyl, —S—C₁₋₃-alkyl, cyclopropyl, —C₁₋₃-alkyl, —C₂₋₃-alkenyl or —C₂₋₃-alkynyl is optionally substituted with at least one substituent selected from halogen, hydroxyl, nitro, cyano and mercapto; with the additional option that any of the substituent pairs, R₁/R₂, R₂/R₃ and R₃/R₄ together with the intervening atoms may form an optionally substituted aromatic ring or ring system;

in the presence of a strong acid (e.g. methanesulfonic acid) so as to provide a compound of formula C

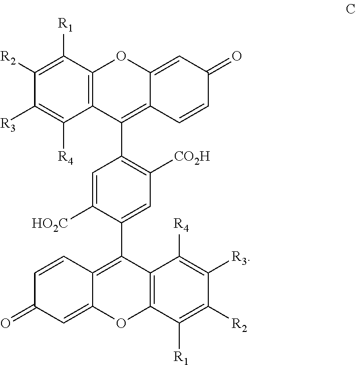

wherein R₁, R₂, R₃ and R₄ are as defined above.

Aspect 8. The method according to any one of aspects 6-7, wherein R₂ and/or R₄ is independently hydroxyl.

Aspect 9. The method according to any one of aspects 6-8, wherein R₁ is halogen, preferably F or Cl.

Aspect 10. The method according to any one of aspects 6-9, wherein R₃ is preferably-O—C₁₋₃-alkyl, such as —OCH₃ or —OC₂H₅.

Aspect 11. The method according to any one of aspects 6-10, wherein A is a dihydroxynaphthalene, preferably 1,3-dihydroxynaphthalene, 2,3-dihydroxynaphthalene, 2,6-dihydroxynaphthalene, 1,4-dihydroxynaphthalene, 1,5-dihydroxynaphthalene, 1,6-dihydroxynaphthalene, 1,8-dihydroxynaphthalene, 1,2-dihydroxynaphthalene, 2,7-dihydroxynaphthalene or 1,7-dihydroxynaphthalene.

Aspect 12. A compound of formula B*

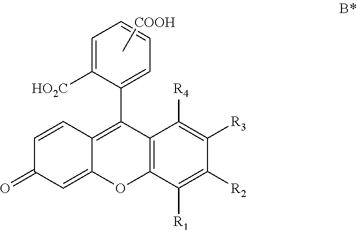

wherein R₁, R₂, R₃ and R₄ are independently selected from hydrogen; halogen; hydroxyl; nitro; cyano; mercapto; —O—C₁₋₃-alkyl; —S—C₁₋₃-alkyl; cyclopropyl; —C₁₋₃-alkyl; —C₂₋₃-alkenyl; or —C₂₋₃-alkynyl; which —O—C₁₋₃-alkyl, —S—C₁₋₃-alkyl, cyclopropyl, —C₁₋₃-alkyl, —C₂₋₃-alkenyl or —C₂₋₃-alkynyl is optionally substituted with at least one substituent selected from halogen, hydroxyl, nitro, cyano and mercapto; with the additional option that any of the substituent pairs, R₁/R₂, R₂/R₃ and R₃/R₄ together with the intervening atoms may form an optionally substituted aromatic ring or ring system.

Aspect 13. A compound according to aspect 12, having the structural formula:

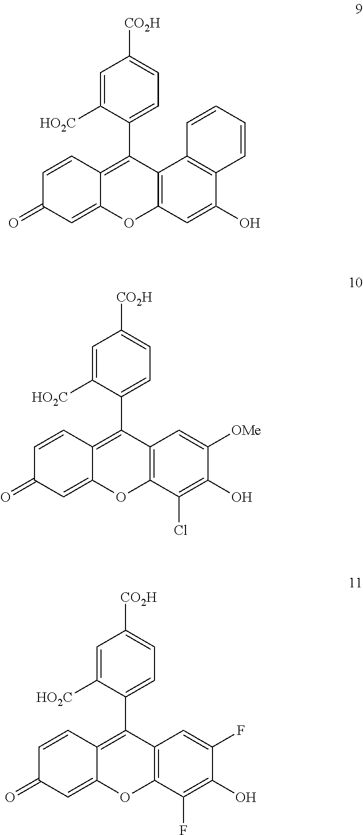 [SMILES: O=C(O)c1ccc(-c2c3ccc(=O)cc-3oc3cc(O)c4ccccc4c23)c(C(=O)O)c1 ; COc1cc2c(-c3ccc(C(=O)O)cc3C(=O)O)c3ccc(=O)cc-3oc2c(Cl)c1O ; O=C(O)c1ccc(-c2c3ccc(=O)cc-3oc3c(F)c(O)c(F)cc23)c(C(=O)O)c1]

Aspect 14. A compound of formula C*

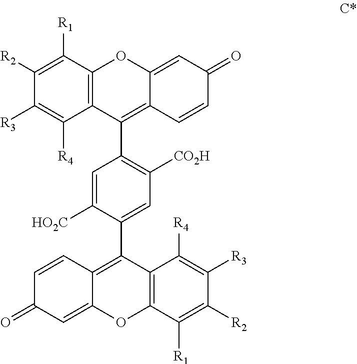

wherein R₁, R₂, R₃ and R₄ are independently selected from hydrogen; halogen; hydroxyl; nitro; cyano; mercapto; —O—C₁₋₃-alkyl; —S—C₁₋₃-alkyl; cyclopropyl; —C₁₋₃-alkyl; —C₂₋₃-alkenyl; or —C₂₋₃-alkynyl; which —O—C₁₋₃-alkyl, —S—C₁₋₃-alkyl, cyclopropyl, —C₁₋₃-alkyl, —C₂₋₃-alkenyl or —C₂₋₃-alkynyl is optionally substituted with at least one substituent selected from halogen, hydroxyl, nitro, cyano and mercapto; with the additional option that any of the substituent pairs, R₁/R₂, R₂/R₃ and R₃/R₄ together with the intervening atoms may form an optionally substituted aromatic ring or ring system.

Aspect 15. A compound according to aspect 14, having the structural formula:

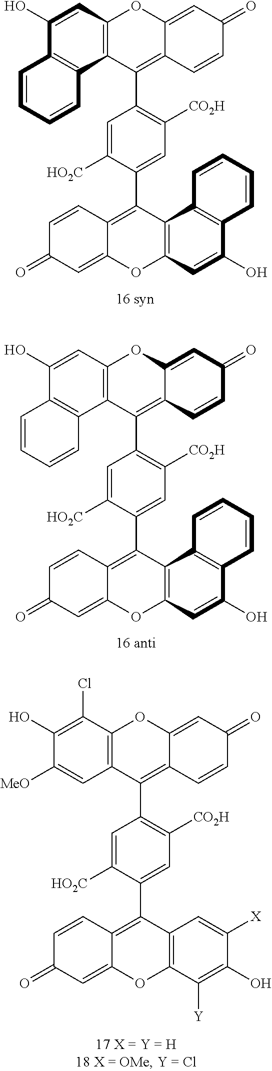

1. A method for the preparation and isolating of compound 6 and, optionally, of compound 5 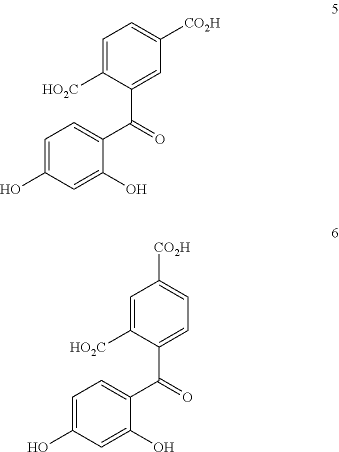 [SMILES: O=C(O)c1ccc(C(=O)O)c(C(=O)c2ccc(O)cc2O)c1 ; O=C(O)c1ccc(C(=O)c2ccc(O)cc2O)c(C(=O)O)c1]
said method comprising the steps of:
(i) providing a condensation product mixture, being the result of a condensation reaction between trimellitic anhydride and resorcinol mediated by acid;
(ii) hydrolysing said condensation product mixture with a strong aqueous base at pH at least 11;
(iii) acidifying the reaction mixture of step (ii) so as to isolate a mixture of compound 5 and compound 6;
(iv) dissolving the mixture of compound 5 and compound 6 in methanol and adding water so as to precipitate compound 6;
(v) extracting the mother liquor with an organic solvent so as to isolate compound 5 and any remaining compound 6, and removing the organic solvent so as to obtain a dried extract;
(vi) optionally repeating steps (iv) and (v) in one or more additional cycles using the dried extract obtained in step (v);
(vii) optionally dissolving the dried extract obtained in step (v) in refluxing H₂O and precipitating compound 5.

2. A method for the preparation and isolation of compound 13 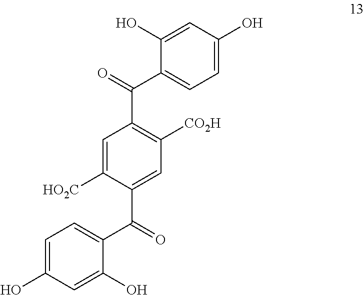 [SMILES: O=C(O)c1cc(C(=O)c2ccc(O)cc2O)c(C(=O)O)cc1C(=O)c1ccc(O)cc1O]
said method comprising the steps of:
(i) providing a condensation product, being the result of a condensation reaction between pyromellitic dianhydride and resorcinol mediated by acid;
(ii) hydrolysing said condensation product with a strong aqueous base at pH of at least 11;
(iii) acidifying the reaction mixture of step (ii) so as to isolate compound 13.

3. The method according to any one of claims 1-2, wherein hydrolysis steps (step ii.) are carried out at a pH of 12-14, preferably using a 1:1 weight ratio mixture of NaOH and H₂O.

4. The method according to any one of claims 1 or 2, wherein the acidification steps (step iii) are carried out using 12 M HCl.

5. The method according to any one of claim 1 or 2, wherein, in step vi, steps (iv) and (v) are repeated in 2-3 additional cycles.

6. The method according to claim 1, wherein compound 5 or compound 6 is subsequently reacted with a compound of the formula A 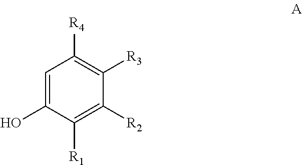
wherein R₁, R₂, R₃ and R₄ are independently selected from hydrogen; halogen; hydroxyl; nitro; cyano; mercapto; —O—C₁₋₆-alkyl; —S—C₁₋₆-alkyl; cyclopropyl; —C₁₋₆-alkyl-CONH—R₅, —C₂₋₆-alkenyl; or —C₂₋₆-alkynyl; which —O—C₁₋₆-alkyl, —S—C₁₋₆-alkyl, cyclopropyl, —C₂₋₆-alkenyl or —C₂₋₆-alkynyl is optionally substituted with at least one substituent selected from halogen, hydroxyl, —COOH, nitro, cyano and mercapto; wherein R₅ is selected from the group consisting of —C₁₋₆-alkyl and —[CH₂CH₂O]ₙ, wherein n=1-10,000, wherein said —C₁₋₆-alkyl and —[CH₂CH₂O]ₙ, are optionally substituted with a substituent selected from the group consisting of —NH-biotin, —C₁₋₆-alkyl-heterocycloalkyl, -DOTA, —NHCO—C₁₋₆-alkyl-heterocycloalkyl, -maleimide, —N₃, —C≡CH, —C-₁₋₆-alkyl-N₃, and —C-₁₋₆-alkyl-N(—C-₁₋₆-alkyl-heteroaryl)₂; with the additional option that any of the substituent pairs, R₁/R₂, R₂/R₃ and R₃/R₄ together with the intervening atoms may form an optionally substituted aromatic ring or ring system;
in the presence of a strong acid (e.g. methanesulfonic acid) so as to provide a compound of formula B 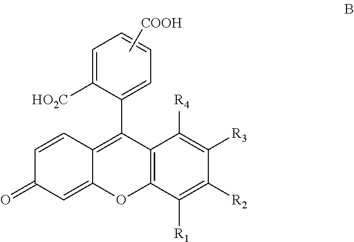
wherein R₁, R₂, R₃ and R₄ are as defined above.

7. The method according to claim 2, wherein compound 13 is subsequently reacted with a compound of the formula A 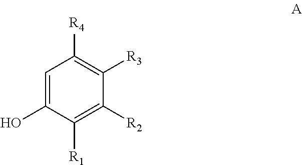
wherein R₁, R₂, R₃ and R₄ are independently selected from hydrogen; halogen; hydroxyl; nitro; cyano; mercapto; —O—C₁₋₆-alkyl; —S—C₁₋₆-alkyl; cyclopropyl; —C₁₋₆-alkyl; —C₁₋₆-alkyl-CONH—R₅, —C₂₋₆-alkenyl; or —C₂₋₆-alkynyl; which —O—C₁₋₆-alkyl, —S—C₁₋₆-alkyl, cyclopropyl, —C₁₋₆-alkyl, —C₂₋₆-alkenyl or —C₂₋₆-alkynyl is optionally substituted with at least one substituent selected from halogen, hydroxyl, —COOH, nitro, cyano and mercapto; wherein R₅ is selected from the group consisting of —C₁₋₆-alkyl and —[CH₂CH₂O]ₙ, wherein n=1-10,000, wherein said —C₁₋₆-alkyl and —[CH₂CH₂O]ₙ are optionally substituted with a substituent selected from the group consisting of —NH-biotin, —C₁₋₆-alkyl-heterocycloalkyl, -DOTA, —NHCO—C₁₋₆-alkyl-heterocycloalkyl, -maleimide, —N₃, —C≡CH, —C-₁₋₆-alkyl-N₃, and —C-₁₋₆-alkyl-N(—C-₁₋₆-alkyl-heteroaryl)₂; with the additional option that any of the substituent pairs, R₁/R₂, R₂/R₃ and R₃/R₄ together with the intervening atoms may form an optionally substituted aromatic ring or ring system;
in the presence of a strong acid (e.g. methanesulfonic acid) so as to provide a compound of formula C 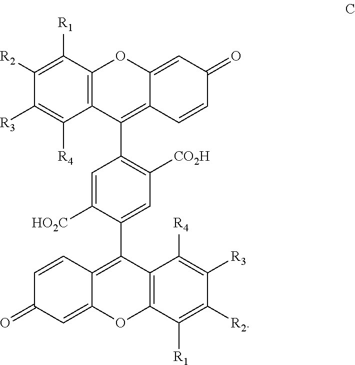
wherein R₁, R₂, R₃ and R₄ are as defined above.

8. The method according to any one of claims 6-7, wherein R₂ and/or R₄ is independently hydroxyl.

9. The method according to any one of claims 6 or 7, wherein R₁ is halogen, preferably F or Cl.

10. The method according to any one of claims 6 or 7, wherein R₃ is —O—C₁₋₃-alkyl, such as —OCH₃ or —OC₂H₅, or —C₁₋₃-alkyl substituted by —COOH, such as —C₂-alkyl substituted by —COOH.

11. The method according to any one of claims 6 or 7, wherein A is a dihydroxynaphthalene, preferably 1,3-dihydroxynaphthalene, 2,3-dihydroxynaphthalene, 2,6-dihydroxynaphthalene, 1,4-dihydroxynaphthalene, 1,5-dihydroxynaphthalene, 1,6-dihydroxynaphthalene, 1,8-dihydroxynaphthalene, 1,2-dihydroxynaphthalene, 2,7-dihydroxynaphthalene or 1,7-dihydroxynaphthalene.

12. A compound of formula B* 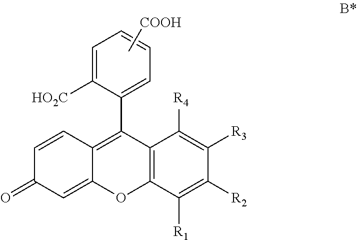
wherein R₁, R₂, R₃ and R₄ are independently selected from hydrogen; fluoro; hydroxyl; nitro; cyano; mercapto; —O—C₁₋₆-alkyl; —S—C₁₋₆-alkyl; cyclopropyl; —C₁₋₆-alkyl; —C₁₋₆-alkyl-CONH—R₅, —C₂₋₆-alkenyl, or —C₂₋₆-alkynyl; which —O—C₁₋₆-alkyl, —S—C₁₋₆-alkyl, cyclopropyl, —C₁₋₆-alkyl, —C₂₋₆-alkenyl or —C₂₋₆-alkynyl is optionally substituted with at least one substituent selected from halogen, hydroxyl, —COOH, nitro, cyano and mercapto; wherein R₅ is selected from the group consisting of —C₁₋₆-alkyl and —[CH₂CH₂O]ₙ, wherein n=1-10,000, wherein said —C₁₋₆-alkyl and —[CH₂CH₂O]ₙ, are optionally substituted with a substituent selected from the group consisting of —NH-biotin, —C₁₋₆-alkyl-heterocycloalkyl, -DOTA, —NHCO—C₁₋₆-alkyl-heterocycloalkyl, -maleimide, —N₃, —C≡CH, —C-₁₋₆-alkyl-N₃, and —C-₁₋₆-alkyl-N(—C-₁₋₆-alkyl-heteroaryl)₂; with the additional option that:
any of the substituent pairs, R₁/R₂, R₂/R₃ and R₃/R₄ together with the intervening atoms may form a substituted aromatic ring or ring system, in which the one or more substituents are selected from halogen; nitro; cyano; mercapto; —O—C₁₋₃-alkyl; —S—C₁₋₃-alkyl; cyclopropyl; —C₁₋₃-alkyl; —C₂₋₃-alkenyl; or —C₂₋₃-alkynyl,
or the compound of formula B* is selected from 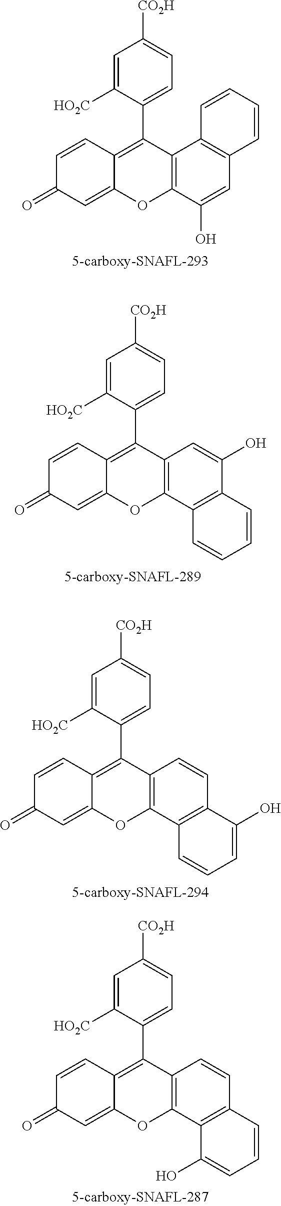 [SMILES: O=C(O)c1ccc(-c2c3ccc(=O)cc-3oc3c(O)cc4ccccc4c23)c(C(=O)O)c1 ; O=C(O)c1ccc(-c2c3ccc(=O)cc-3oc3c2cc(O)c2ccccc23)c(C(=O)O)c1 ; O=C(O)c1ccc(-c2c3ccc(=O)cc-3oc3c2ccc2c(O)cccc23)c(C(=O)O)c1 ; O=C(O)c1ccc(-c2c3ccc(=O)cc-3oc3c2ccc2cccc(O)c23)c(C(=O)O)c1]
with the proviso that when R₁=R₃=R₄=hydrogen, then R₂ is different from hydroxyl.

13. A compound according to claim 12, having the structural formula:

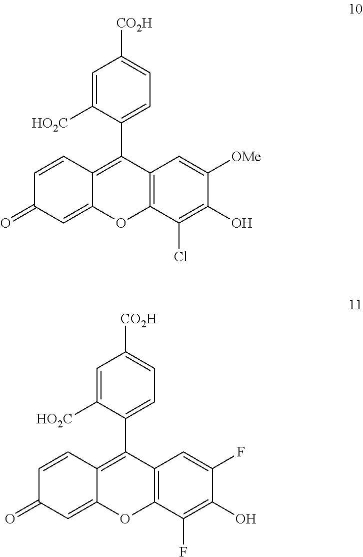 [SMILES: COc1cc2c(-c3ccc(C(=O)O)cc3C(=O)O)c3ccc(=O)cc-3oc2c(Cl)c1O ; O=C(O)c1ccc(-c2c3ccc(=O)cc-3oc3c(F)c(O)c(F)cc23)c(C(=O)O)c1]

14. A compound of formula C* 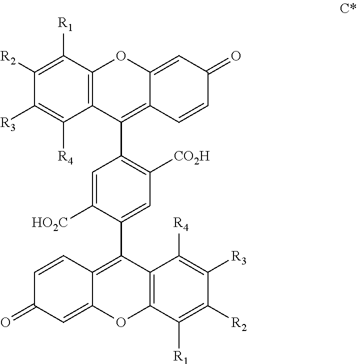
wherein R₁, R₂, R₃ and R₄ are independently selected from hydrogen; halogen; hydroxyl; nitro; cyano; mercapto; —O—C₁₋₆-alkyl; —S—C₁₋₆-alkyl; cyclopropyl; —C₁₋₆-alkyl; —C₁₋₆-alkyl-CONH—R₅, —C₂₋₆-alkenyl; or —C₂₋₆-alkynyl; which —O—C₁₋₆-alkyl, cyclopropyl, —C₁₋₆-alkyl, —C₂₋₆-alkenyl or —C₂₋₆-alkynyl is optionally substituted with at least one substituent selected from halogen, hydroxyl, —COOH, nitro, cyano and mercapto; wherein R₅ is selected from the group consisting of —C₁₋₆-alkyl and —[CH₂CH₂O]ₙ, wherein n=1-10,000, wherein said —C₁₋₆-alkyl and —[CH₂CH₂O]ₙ are optionally substituted with a substituent selected from the group consisting of —NH-biotin, —C₁₋₆-alkyl-heterocycloalkyl, -DOTA, —NHCO—C₁₋₆-alkyl-heterocycloalkyl, -maleimide, —N₃, —C≡CH, —C-₁₋₆-alkyl-N₃, and —C-₁₋₆-alkyl-N(—C-₁₋₆-alkyl-heteroaryl)₂; with the additional option that any of the substituent pairs, R₁/R₂, R₂/R₃ and R₃/R₄ together with the intervening atoms may form an optionally substituted aromatic ring or ring system, with the proviso that when R₁=R₃=R₄=hydrogen, then R₂ is different from hydroxyl.

15. A compound according to claim 14, having the structural formula:

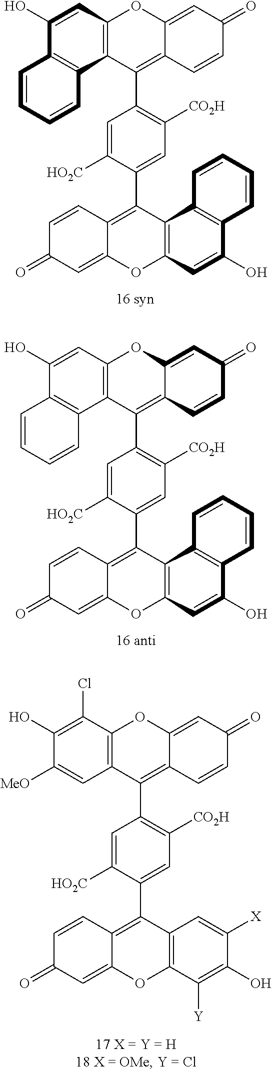

16. A compound of the formula 5 or 6 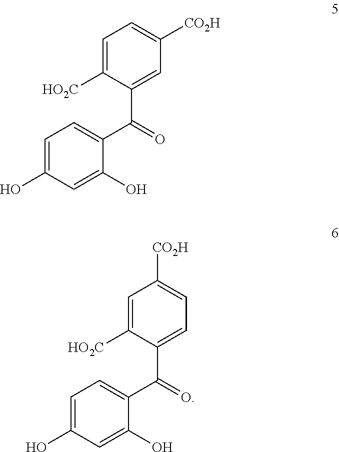 [SMILES: O=C(O)c1ccc(C(=O)O)c(C(=O)c2ccc(O)cc2O)c1 ; O=C(O)c1ccc(C(=O)c2ccc(O)cc2O)c(C(=O)O)c1]
